(19) United States
(12) Patent Application Publication
(10) Pub. No.: US 2015/0105328 A1
(43) Pub. Date: Apr. 16, 2015

(54) PEPTOID-PEPTIDE HYBRIDS AND THEIR USE

(71) Applicants: UNIVERSITY OF SOUTH FLORIDA, TAMPA, FL (US); H. LEE MOFFITT CANCER CENTER AND RESEARCH INSTITUTE, INC., TAMPA, FL (US); MODULATION THERAPEUTICS, TAMPA, FL (US)

(72) Inventors: MARK MCLAUGHLIN, TAMPA, FL (US); PRIYESH JAIN, TAMPA, FL (US)

(21) Appl. No.: 14/574,776

(22) Filed: Dec. 18, 2014

Related U.S. Application Data
(63) Continuation of application No. PCT/US2013/047417, filed on Jun. 24, 2013.
(60) Provisional application No. 61/663,325, filed on Jun. 22, 2012.

(51) Int. Cl. C07K 7/54; A61K 45/06; G01N 33/50; A61K 38/12

(52) U.S. Cl. CPC C07K 7/54; A61K 38/12; A61K 45/06; G01N 33/5011; G01N 33/5008; G01N 2500/10; G01N 2510/00
USPC 514/19.9; 530/317; 514/21.1; 506/18; 506/9; 435/375

(57) ABSTRACT

The invention concerns peptoid-peptide hybrids that may act as protein interaction inhibitors (PPIIs). Another aspect of the invention concerns a method for treating a disorder (e.g., an oncologic disorder) in a human or animal subject, comprising administering an effective amount of a peptoid-peptide hybrid (a peptoid body) of the invention, or a composition comprising a peptoid-peptide hybrid, to the subject in need thereof. Another aspect of the subject invention concerns a method for killing or inhibiting the growth of cells (e.g., cancer cells or malaria-infected cells), comprising contacting a cancer cell in vitro or in vivo with an effective amount of a peptoid-peptide hybrid, or a composition comprising the peptoid-peptide hybrid. Another aspect of the invention concerns a method for producing a peptoid-peptide hybrid.

### CROSS-REFERENCE TO RELATED APPLICATIONS

The present application is a continuation of International Application No. PCT/US2013/047417, filed Jun. 24, 2013, which claims the benefit of U.S. Provisional Application Ser. No. 61/663,325, filed Jun. 22, 2012, each of which is hereby incorporated by reference herein in its entirety, including any figures, tables, nucleic acid sequences, amino acid sequences, or drawings.

### GOVERNMENT SUPPORT

This invention was made with government support under grant number CA118210 awarded by the National Institutes of Health. The government has certain rights in the invention.

### BACKGROUND OF THE INVENTION

Protein-protein interactions (PPIs) are increasingly important drug targets and this assertion is strongly supported by the statistic that half of the 12 branded blockbuster drugs for 2009 are biologics that inhibit PPIs. These 12 blockbuster drugs are those with worldwide sales in excess of $5 billion/yr, which is a surrogate marker for a recently met medical need. Three of these biologics (ENBREL, REMICADE, and HUMIRA) bind to tumor necrosis factor-alpha (TNF-alpha) and block its interaction with the TNF-alpha receptor; this PPI inhibition reduces pathological inflammation in autoimmune diseases, such as rheumatoid arthritis, Crohn's disease, and others. The remaining three blockbuster drug biologics (AVASTIN, RITUXAN, and HERCEPTIN) are therapeutic antibodies used to treat cancer. AVASTIN binds to vascular endothelial growth factor A (VEGF-A) and this inhibits angiogenesis because VEGF-A is a protein messenger for the growth of new capillaries and a vigorous new blood supply enables the growth of spreading cancer. RITUXAN binds to CD20 receptors on the surface of B-cells; it kills both malignant and normal B cells and so it is used to treat leukemias, lymphomas, and autoimmune diseases that do not respond to safer alternative treatments. HERCEPTIN inhibits the activity of the HER2/neu receptor, which is often overexpressed on the surface of breast cancer cells; the breast cancer cells depend on downstream signals from the HER2/neu receptor more than normal breast cells and kill or slow the growth of the targeted breast cancer. This recent trend of biological drug development is expected to continue and by 2014, it is estimated that 50% of the 100 top selling drugs will be biologics and almost all of these will work by inhibiting PPIs. The market leaders among these will be anti-cancer therapeutic antibodies that will work like those mentioned above, but that target some other specific extracellular or secreted protein.

Unfortunately, biologics are very expensive to manufacture relative to typical small molecule drugs and their large complex structures can lead to unintended side effects. For instance, REMICADE is a chimeric monoclonal antibody (combination of a mouse and human antibody) and some patients develop an allergic reaction that causes life-threatening breathing problems. Even for fully humanized proteins, the immune system, upon repeated exposure to the biologic, can develop neutralizing antibodies and reduce drug efficacy. More importantly, the large size of biologics inhibits cell permeability and so they only effectively target extracellular proteins or secreted proteins.

As antibodies are incredibly diverse with about 10 billion different hypervariable regions in a human with a level of molecular diversity usually gives the epitope-antibody binding affinities and specificities that make antibodies so useful. However, it is impractical to make a synthetic protein-like scaffold library with 10 billion or more different members. Therefore, a library that has the possibility of scanning that level of molecular diversity is desirable.

### BRIEF SUMMARY OF THE INVENTION

A library of peptoid bodies is disclosed herein, which permits scanning of an enormous level of molecular diversity. The peptoid bodies are cyclic peptoid-peptide hybrids, which can provide a scaffold library using a pairwise combinatorial approach. Embodiments of the invention are directed to cyclic peptoid-peptide hybrids that adopt a beta-hairpin-like secondary structure. This cyclic beta-hairpin-like design results from the alternation of the peptide-peptoid sub-units in two antiparallel beta-strands. The cyclic peptoid-peptide hybrids have the chemical structure, for example, shown in formula I:

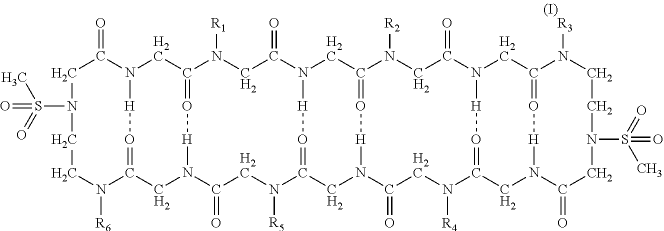

or a pharmaceutically acceptable salt or hydrate thereof, wherein R₁-R₆ are independently organic groups.

In an embodiment of the invention, the cyclic peptoid-peptide hybrids have the chemical structure shown in formula II:

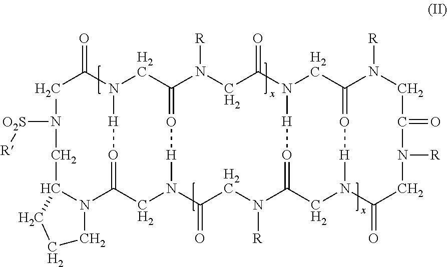

or a pharmaceutically acceptable salt or hydrate thereof, wherein R groups are independently organic groups, R′ is an organic group, or an organic bridging group to a resin or other substrate, and x is 1 to 3.

In an embodiment of the invention, the cyclic peptoid-peptide hybrids have the chemical structure shown in formula III:

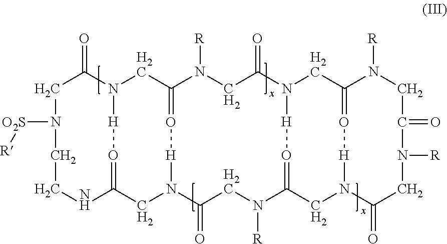

or a pharmaceutically acceptable salt or hydrate thereof, wherein R groups are independently organic groups, R′ is an organic group, or an organic bridging group to a resin or other substrate, and x is 1 to 3.

In an embodiment of the invention, the cyclic peptoid-peptide hybrids have the chemical structure shown in formula IV:

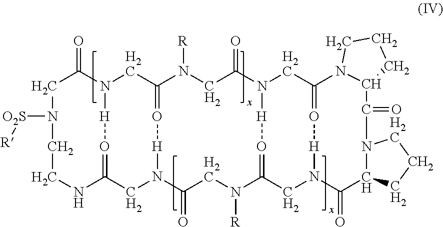

or a pharmaceutically acceptable salt or hydrate thereof, wherein R groups are independently organic groups, R′ is an organic group, or an organic bridging group to a resin or other substrate, and x is 1 to 3.

In an embodiment of the invention, the cyclic peptoid-peptide hybrids have the chemical structure shown in formula V:

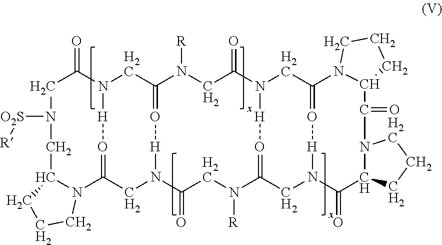

or a pharmaceutically acceptable salt or hydrate thereof, wherein, R groups are independently organic groups; R′ is an organic group, or an organic bridging group to a resin or other substrate, and x is 1 to 3.

In an embodiment of the invention, the cyclic peptoid-peptide hybrids have the chemical structure shown in formula VI:

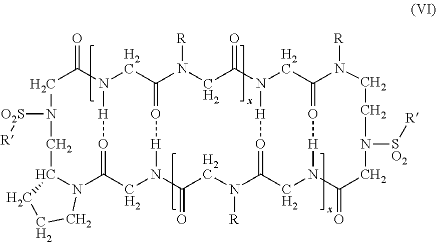

or a pharmaceutically acceptable salt or hydrate thereof, wherein R groups are independently organic groups, R′ is independently an organic group, or an organic bridging group to a resin or other substrate, and x is 1 to 3.

In an embodiment of the invention, the cyclic peptoid-peptide hybrids have the chemical structure shown in formula VII:

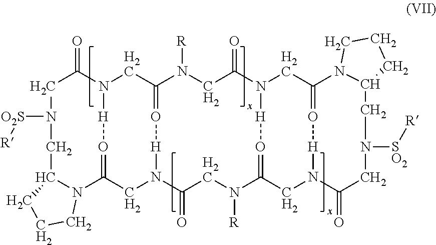

or a pharmaceutically acceptable salt or hydrate thereof, wherein R groups are independently organic groups, R′ is independently an organic group, or an organic bridging group to a resin or other substrate, and x is 1 to 3.

In an embodiment of the invention, the cyclic peptoid-peptide hybrids have the chemical structure shown in formula VIII:

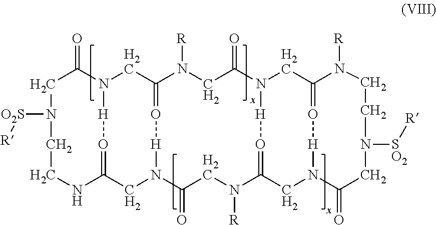

or a pharmaceutically acceptable salt or hydrate thereof, wherein R groups are independently organic groups, R′ is independently an organic group, or an organic bridging group to a resin or other substrate, and x is 1 to 3.

In an embodiment of the invention, the cyclic peptoid-peptide hybrids have the chemical structure shown in formula IX:

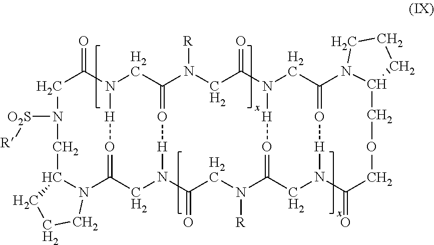

or a pharmaceutically acceptable salt or hydrate thereof, wherein R groups are independently organic groups, R′ is an organic group, or an organic bridging group to a resin or other substrate, and x is 1 to 3.

In an embodiment of the invention, the cyclic peptoid-peptide hybrids have the chemical structure shown in formula X:

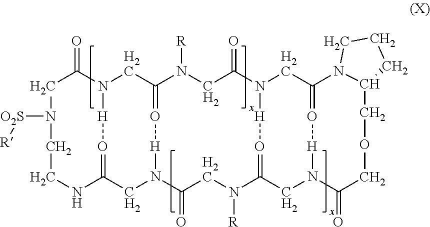

or a pharmaceutically acceptable salt or hydrate thereof, wherein R groups are independently organic groups, R′ is an organic group, or an organic bridging group to a resin or other substrate, and x is 1 to 3.

In an embodiment of the invention R groups of at least two adjacent peptoid-glycine sequences are 4-piperidinyl groups, for example, a compound of Formula II where x is 2:

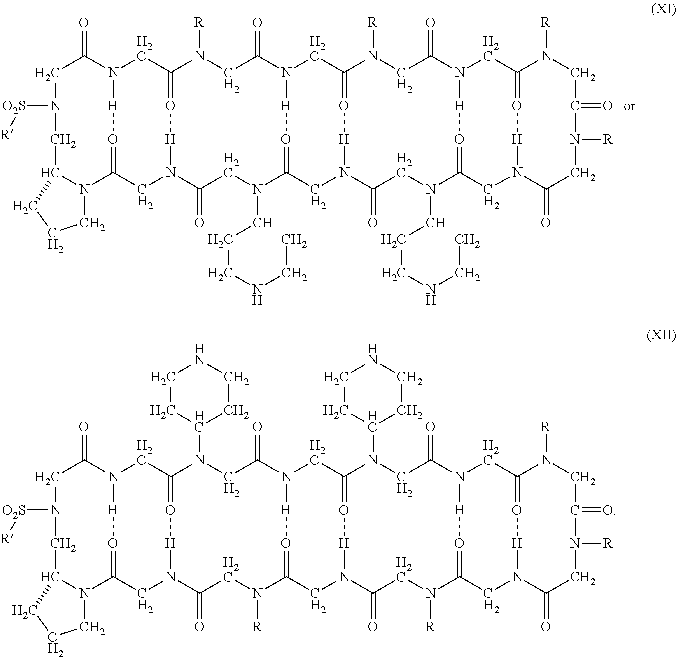

### BRIEF DESCRIPTION OF THE DRAWINGS

FIG. 1 shows representative peptoid-bodies, according to an embodiment of the invention.

FIG. 2 shows a schematic for the preparation of a peptoid-body comprising a linker that is bound to a resin and a second linker comprising a peptoid-peptoid sequence, according to an embodiment of the invention.

FIG. 3 shows a schematic for the preparation of a peptoid-body comprising a linker that is bound to a resin and a second linker that is a beta-turn promoter, according to an embodiment of the invention.

FIGS. 4A and 4B show a photograph of a polypropylene reservoir with a 300-micron chlorosulfonic acid modified PEEK film (FIG. 4A), for formation of a peptoid-body array, according to an embodiment of the invention, and a 5× magnification of the reservoir (FIG. 4B) where amino-tentagel resin beads are attached to the PEEK film, according to an embodiment of the invention.

### DETAILED DISCLOSURE OF THE INVENTION

Peptoid-bodies are small, synthetic protein-like scaffolds that can identify differentially expressed extracellular proteins and hence define novel therapeutic targets. Peptoid-bodies contain variable side chains within a well-defined secondary structure, as in the hypervariable regions of antibodies. Like standard peptoid combinatorial libraries, the peptoid-bodies, according to embodiments of the invention, can display very diverse side chains, but, unlike standard peptoid libraries, a cyclic beta-hairpin-like conformation is adapted. Embodiments of the invention are directed to “peptoid-bodies” that are cyclic peptoid-peptide hybrids which contain a beta-hairpin-like secondary structure. The cyclic peptoid-peptide hybrids display a plurality of peptoid-peptide sequences of an alternation of a peptoid, where a side chain is connected to the nitrogen of the peptide backbone, rather than the α-carbon of peptides, and an amino acid residue, for example, a glycine residue. In this manner, the peptoid portion provides resistant to proteolysis and the peptide portion of the peptoid-peptide hybrids provides the ability to achieve the beta-hairpin-like secondary structure. These two contributions result in a hybrid that is a good drug candidate for therapies where proteolysis is generally a limitation of the therapy.

This cyclic beta-hairpin-like design results from alternating peptide-peptoid sub-units that reside as two antiparallel beta-strands, as can be seen in Formula I, below, where R₁ through R₆ can be organic groups from any of the 230 different amines that have been used to form peptoids. There cyclic peptoid-peptide hybrids comprise a large diverse chemical library that can be constructed at a low cost relative to similar peptides libraries due to the thousands of commercially available amines that provide a broad chemical diversity at each peptoid position.

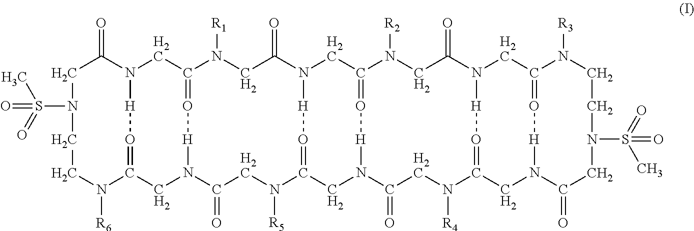

Embodiments of the invention concern compositions comprising one or more of the cyclic peptoid-peptide hybrids disclosed herein. In an embodiment of the invention, the cyclic peptoid-peptide hybrids constitute agents for use in a method of treating a disorder in a human or animal subject, comprising administering an effective amount of a cyclic peptoid-peptide hybrid or a composition comprising the cyclic peptoid-peptide hybrid, to the subject in need of treatment. An embodiment of the invention concerns a method for killing or inhibiting the growth of cancer cells, comprising contacting a cancer cell in vitro or in vivo with an effective amount of a cyclic peptoid-peptide hybrid or a composition comprising the hybrid peptoid-peptide inhibitor.

An embodiment of the invention concerns a method for producing a cyclic peptoid-peptide hybrid compound, comprising selecting a peptoid-glycine sequence and synthesizing the cyclic compound, wherein said cyclic compound contains the selected alternating peptoid-glycine sequence. The cyclic peptoid-peptide hybrid compound comprising a first linker moiety (a first beta-turn promoter) and a second linker moiety (a second beta-turn promoter), which may be the same or different. The peptoid-glycine sequences between the first and second linker moieties form antiparallel strands that hydrogen bond to form a scaffold. The terms “linker” and “linker moiety” are used interchangeably herein to refer to a beta-turn promoter. As shown in Formula II, below, a peptoid-peptoid sequence may provide a second linker.

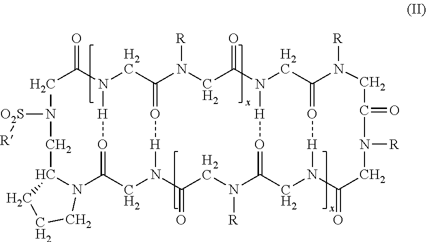

wherein R groups are independently organic groups, R′ is an organic group, or an organic bridging group to a resin or other substrate, and x is 1 to 3.

In embodiments of the invention, at least one linker moiety is a beta turn promoter. The beta turn promoter can be an amino acid residue from the condensation of the linker precursor of the structure T₁, T₂, T₃ or T₄, below, or any other linker precursor that promotes a beta turn.

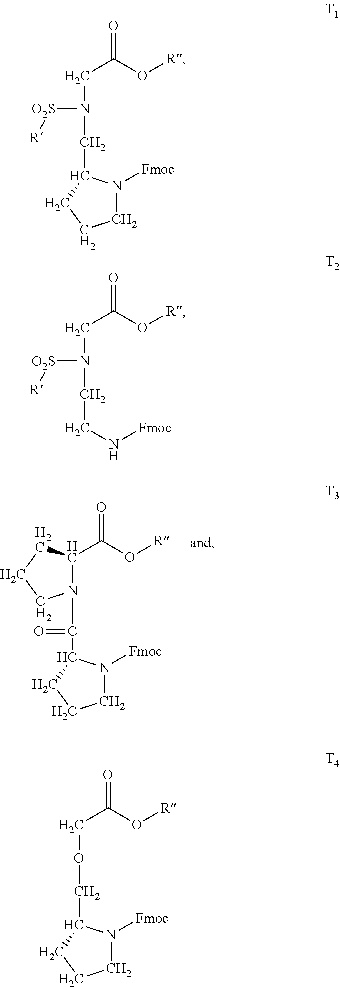

where R′ is an organic group, or an organic bridging group to a resin or other substrate and R″ is H, t-butyl, allyl, benzyl, or other carboxylic acid protecting group. All exemplary cyclic peptoid-peptide hybrids are illustrated herein with at least one T₁ or T₂ groups. Advantageously, according to an embodiment of the invention, where these linkers include an R′ group that is an organic bridging group to a resin or other substrate, the substrate can be employed to form and bind at least one peptoid body to a substrate for synthesis and/or bound for screening the affinity of peptoid bodies in a library to target entities. Upon discovery of an active agent for a therapy using a substrate (e.g., a resin), as desired, a substrate-free (e.g., resin-free) equivalent can be generated by changing the R′ unit to a methyl or other small organic group. Other exemplary peptoid bodies, according to an embodiment of the invention can have a structure as shown in Formula II through X, below, wherein R groups are independently organic groups, R′ is independently an organic group or an organic bridging group to a resin or other substrate, and x is 1 to 3:

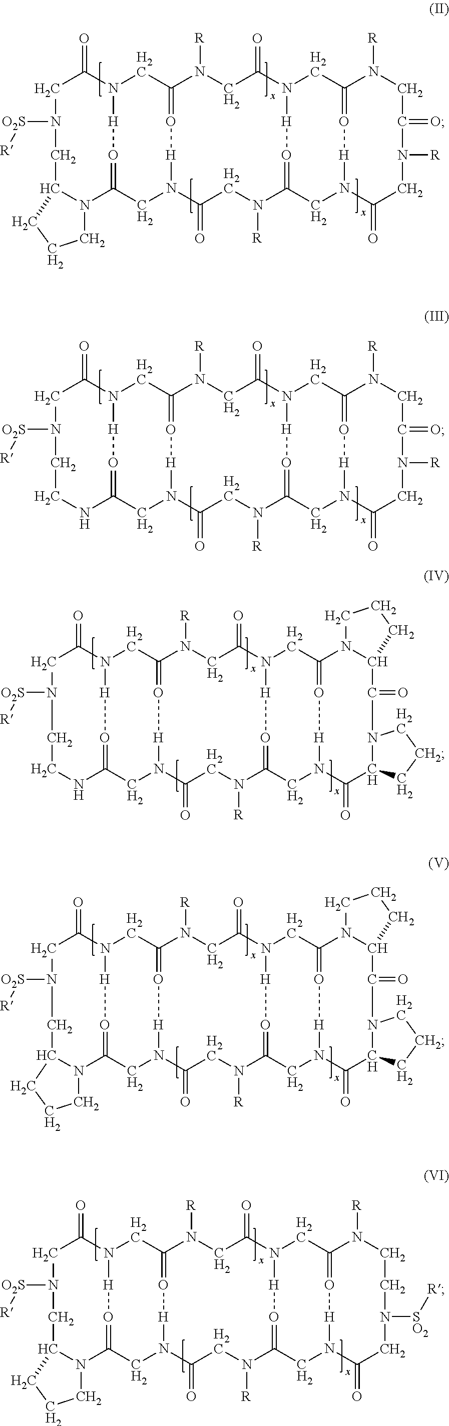

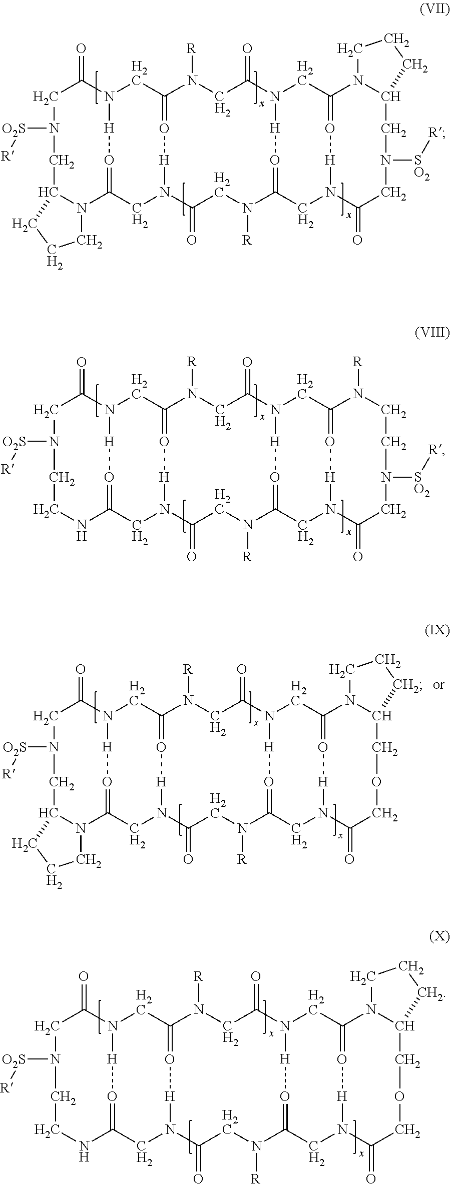

The R groups of the cyclic peptoid-peptide hybrids of Formula I through X, above, and Formula XI and XII, below, can be of almost any structure with a primary amine functionality that does not disrupt the complementary hydrogen bonding of the beta sheet structure within the cyclic peptoid-peptide hybrid. The R group can be equivalent to the side chains of amino acids, the non-amine portion of an amino acid, or modified non-amine portion of an amino acid. The R group can be a sugar, such as, a mono-saccharide or di-saccharide, or a fatty acid, or modified variation thereof. The R group can be, but is not limited to, C₁-C₁₂ alkyl, C₁-C₁₂ hydroxyalkyl, C₁-C₁₂ aminoalkyl, C₁-C₁₂ carboxylic acid alkyl, C₂-C₁₂ alkyloxyalkyl, C₂-C₁₂ alkenyl, C₂-C₁₂ hydroxyalkenyl, C₂-C₁₂ aminoalkenyl, C₁-C₁₂ carboxylic acid alkenyl, C₃-C₁₄ alkyloxyalkenyl, C₆-C₁₄ aryl, C₆-C₁₄ hydroxyaryl, C₆-C₁₄ aminoaryl, C₆-C₁₄ carboxylic acid aryl, C₇-C₁₅ alkyloxyaryl, C₄-C₁₄ heteroaryl, C₄-C₁₄ hydroxyheteroaryl, C₄-C₁₄ aminoheteroaryl, C₄-C₁₄ carboxylic acid heteroaryl, C₅-C₁₅ alkoxyheteroaryl, C₇-C₁₅ alkylaryl, C₇-C₁₅ hydroxyalkylaryl, C₇-C₁₅ aminoalkylaryl, C₇-C₁₅ carboxylic acid alkylaryl, C₈-C₁₅ alkoxyalkylaryl, or a chemically transformed product of any of these R groups, such as, esters, thioesters, thiols, amides, or sulfonamides, wherein alkyl groups can be linear, branched, multiply branched, cyclic, or polycyclic. For example, R can be, a residue of a primary amine, which can be, but is not limited to, groups from the incorporation of 4-aminopiperidine, ethanolamine, allylamine, 1,4-diaminobutane, piperponylamine, 4,(2-aminoethyl)benzene, isobutylamine, tryptamine, 4-morpholinoaniline, 5-amino-2-methoxypyridine, (R)-methylbenzylamine, 1-(2-aminopropyl)-2-pyrrolidinone, furfurylamine, benzylamine, 4-chlorobenzylamine, 4-methoxybenzylamine, methoxyethylamine, 2-aminoadipic acid, N-ethylasparagine, 3-aminoadipic acid, hydroxylysine, beta-alanine, allo-hydroxylysine propionic acid, 2-aminobutyric acid, 3-hydroxyproline, 4-Aminobutyric acid, 4-hydroxyproline piperidinic acid, 6-aminocaproic acid, isodesmosine, 2-aminoheptanoic acid, allo-isoleucine, 2-aminoisobutyric acid, N-methylglycine, 3-aminoisobutyric acid, N-methylisoleucine, 2-Aminopimelic acid, 6-N-methyllysine, 2,4-diaminobutyric acid, N-methylvaline, desmosine, norvaline, 2,2′-diaminopimelic acid, norleucine, 2,3-diaminopropionic acid, ornithine, N-ethylglycine, or protected equivalents thereof, as a peptoid N—R unit in the cyclic peptoid-peptide hybrid.

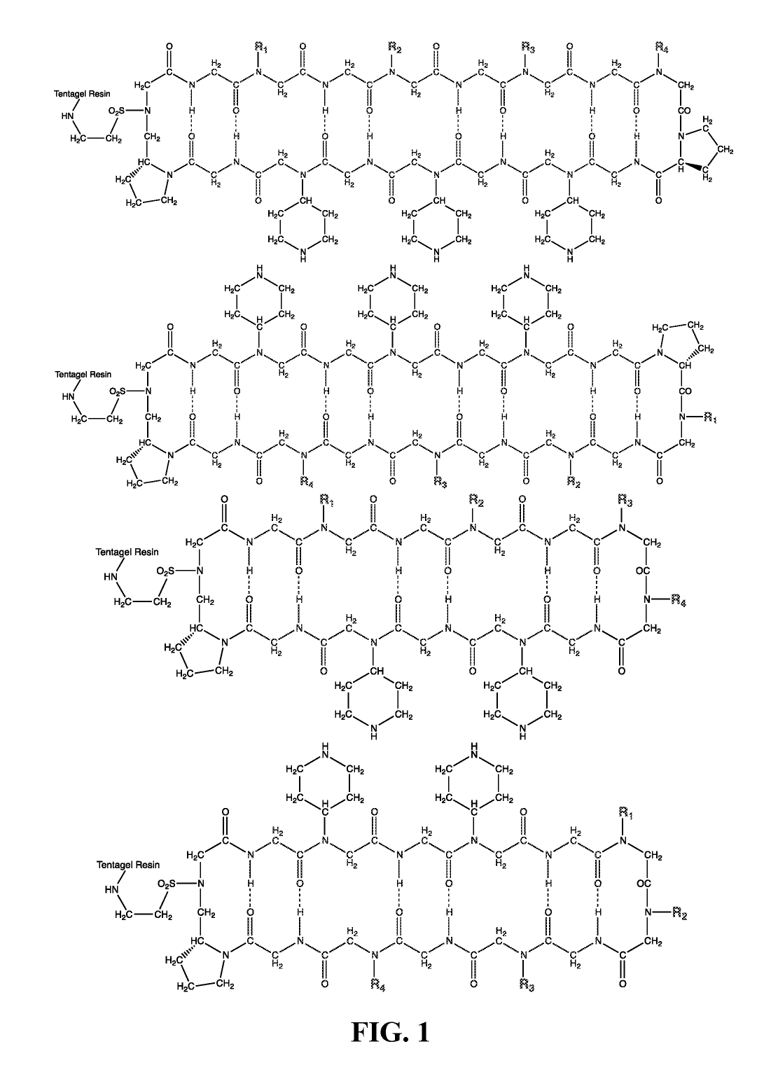

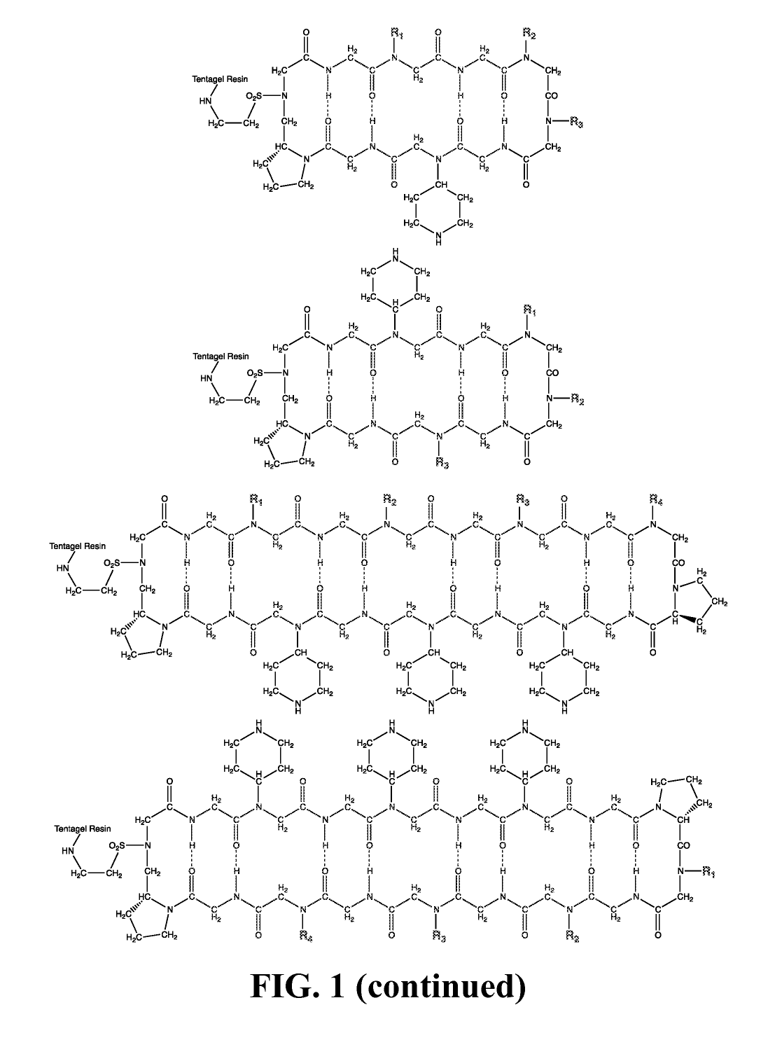

According to an embodiment of the invention, a plurality of the peptoid bodies can be produced that are bound to substrates such as resins and used to identify differentially expressed extracellular proteins that are associated with a diseased cell versus an untargeted cell. For example, libraries of 714,150 members can be generated to identify novel cell surface proteins present (or over-expressed) on cancer cells. With a peptoid body having the structure of Formula XI or XII, below, where R′ is, for example, a resin attached via a —NH(CH₂)₂— bridge, a library can be prepared that displays 345 different side chains R in two of four variable peptoid positions to give 119,025 different library members (345×345) times 6 possible pairwise combinations result in 714,150 library members. The bound top-binding hits in these screens will be left unchanged and the 2 additional peptoid positions can display 345 different side chains, each with another 119,025 different library members per hit. The top hits can subsequently undergo single side-chain replacement to optimize binding further. Those scaffolds display a molecular diversity selected for binding to the target, where out of more than 14 billion possible structures, optimal agents can be identified form a full combinatorial library of only about 1.4 million (345⁴) different members. Upon production of a library, according to an embodiment of the invention, a spatially addressable parallel library, with an array of spots having cleavable and stable linkages to the solid-phase substrate with a 384-well-like format. The 384-well-like array allows automated synthesis, plate washing and plate screening using existing robotic equipment that is available in many research facilities. Other peptoid bodies with at least one 4-piperidenyl R groups are shown in FIG. 1.

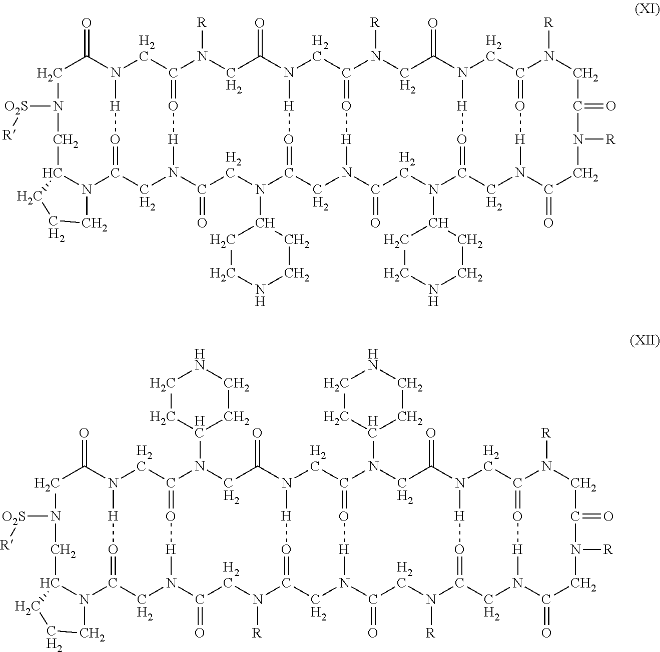

Subsequently, identification of therapeutic targets of cancers, for example, but not limited to: chemotherapy resistant triple negative breast cancer; platinum-resistant ovarian cancer (OVCA); lung cancer stem cells isolated by cell sorting, using ALDH1 and side-population phenotypes; prostate bone metastases; or any other metastatic tumor cells. For example, OVCA platinum-resistant cells can be labeled with red fluorescent quantum dots and OVCA platinum-sensitive parent cells with green fluorescent quantum dots. Using a two-color sensing automated fluorescence multi-plate reader the library is screen for hits that differentially bind the red OVCA platinum-resistant cells. The hits that bind only the red cells have an extracellular target that is only expressed or overexpressed on the platinum-resistant OVCA cells. Subsequently, the peptoid bodies representing hits are re-synthesized to verify its specific binding to the platinum-resistant versus platinum-sensitive OVCA cells. Subsequently, a biotinylated version of the hit peptoid body is prepared and any molecules bound to the biotinylated hit peptide body are analyzed using proteomics methods. Typically, the bound molecules will usually be a complex mixture of the direct binding partner and whatever other proteins that are laterally associated with the direct binding partner. In parallel, the hit is photoaffinity labeled for performance of photo-tagging of the direct binding protein and denaturing conditions are established to break up noncovalent protein-protein aggregates. Determination of the direct-hit molecule binding partner is then carried out using proteomic analysis. Where the cell surface targets are among those reported targets as biomarkers of OVCA platinum-resistant; the recombinant protein will be used to verify that the library screening hit binds that recombinant protein. The screening permits the discovery of unknown biomarkers of OVCA platinum-resistance.

The peptoid body approach has the potential to change disease diagnoses and treatment, as an array of peptoid bodies can be used to repeatedly screen a wide range of targets followed by identification of easily produced targeting ligands economically. Table 1, below, provides a non-exclusive list of possible targeted and untargeted cells.

TABLE 1
Targeted and Untargeted Cells

| Possible Targeted Cell | Possible Untargeted Cell |
|---|---|
| Multi-drug resistant bacteria (>50 possible | The same bacteria that has not acquired |
| pathogens) | resistance |
| Find a targeted agent that only kills the bad | |
| strains of E coli and spares the harmless E | |
| coli or other harmless strains of bacteria | |
| Human cell with an intracellular pathogen | The same human cell lacking the infection |
| (latent TB, latent HIV, malaria etc.) | |
| An acquired drug-resistant cancer cell | The same cancer cell before it has acquired |
| (Lung, Prostate, Breast, etc.) | drug-resistance |
| Autoimmune diseases - Isolate effector- | Normal effector-cells from a person |
| cells from persons with RA, Crohn's etc. | lacking an autoimmune disease and label |
| and label them red | them green - a close blood relative would |
| Treat subjects with peptoid-body hits that | reduce spurious binding. |
| only bind the autoreactive T-cells | |
| Potentially prevent Alzheimer's - Apo-E-4 | Apo-E-3 labeled green |
| labeled red Find a specific ligand to block | |
| apo-E-4 interaction with the abeta peptide | |
| but that does not block Apo-E-3 interaction | |
| with the abeta peptide. | |
| Replace almost any currently used antibody | A closely related epitope labeled green |
| by labeling the desired epitope with a red | |
| tag | |

An aspect of the invention includes a method for treating a disorder in a subject, comprising administering an effective amount of a pharmaceutical composition of the invention to the subject. The peptide-peptoid hybrids can be screened to bind any protein; therefore, it should be appreciated that any disorder in which targeted killing, or targeted payloads, or interference with binding of two molecules is therapeutic, may be treated.

Various disorders may be treated, such as an oncological disorders (cancers), infections (e.g., from a pathogenic microorganism such as malaria), immunoregulatory abnormalities, such as autoimmune diseases and chronic inflammatory diseases. Examples of autoimmune or chronic inflammatory diseases include amyotrophic lateral sclerosis (ALS), systemic lupus erythematosus, chronic rheumatoid arthritis, type I diabetes mellitus, inflammatory bowel disease, biliary cirrhosis, uveitis, multiple sclerosis, Crohn's disease, ulcerative colitis, bullous pemphigoid, sarcoidosis, psoriasis, autoimmune myositis, Wegener's granulomatosis, ichthyosis, Graves ophthalmopathy and asthma. Other examples of immunoregulatory abnormality are bone marrow or organ transplant rejection or graft-versus-host disease. In some embodiments, the immunoregulatory abnormality is selected from the group consisting of: rejection brought about transplantation of organs or tissue, graft-versus-host diseases brought about by transplantation, autoimmune syndromes including rheumatoid arthritis, systemic lupus erythematosus, Hashimoto's thyroiditis, multiple sclerosis, myasthenia gravis, type I diabetes, uveitis, posterior uveitis, allergic encephalomyelitis, glomerulonephritis, post-infectious autoimmune diseases including rheumatic fever and post-infectious glomerulonephritis, inflammatory and hyperproliferative skin diseases, psoriasis, atopic dermatitis, contact dermatitis, eczematous dermatitis, seborrhoeic dermatitis, lichen planus, pemphigus, bullous pemphigoid, epidermolysis bullosa, urticaria, angioedemas, vasculitis, erythema, cutaneous eosinophilia, lupus erythematosus, acne, alopecia greata, keratoconjunctivitis, vernal conjunctivitis, uveitis associated with Behcet's disease, keratitis, herpetic keratitis, conical cornea, dystrophia epithelialis corneae, corneal leukoma, ocular pemphigus, Mooren's ulcer, scleritis, Graves' opthalmopathy, Vogt-Koyanagi-Harada syndrome, sarcoidosis, pollen allergies, reversible obstructive airway disease, bronchial asthma, allergic asthma, intrinsic asthma, extrinsic asthma, dust asthma, chronic or inveterate asthma, late asthma and airway hyper-responsiveness, bronchitis, gastric ulcers, vascular damage caused by ischemic diseases and thrombosis, ischemic bowel diseases, inflammatory bowel diseases, necrotizing enterocolitis, intestinal lesions associated with thermal burns, coeliac diseases, proctitis, eosinophilic gastroenteritis, mastocytosis, Crohn's disease, ulcerative colitis, migraine, rhinitis, eczema, interstitial nephritis, Goodpasture's syndrome, hemolytic-uremic syndrome, diabetic nephropathy, multiple myositis, Guillain-Barre syndrome, Meniere's disease, polyneuritis, multiple neuritis, mononeuritis, radiculopathy, hyperthyroidism, Basedow's disease, pure red cell aplasia, aplastic anemia, hypoplastic anemia, idiopathic thrombocytopenic purpura, autoimmune hemolytic anemia, agranulocytosis, pernicious anemia, megaloblastic anemia, anerythroplasia, osteoporosis, sarcoidosis, fibroid lung, idiopathic interstitial pneumonia, dermatomyositis, leukoderma vulgaris, ichthyosis vulgaris, photoallergic sensitivity, cutaneous T cell lymphoma, chronic lymphocytic leukemia, arteriosclerosis, atherosclerosis, aortitis syndrome, polyarteritis nodosa, myocardosis, scleroderma, Wegener's granuloma, Sjogren's syndrome, adiposis, eosinophilic fascitis, lesions of gingiva, periodontium, alveolar bone, substantia ossea dentis, glomerulonephritis, male pattern alopecia or alopecia senilis by preventing epilation or providing hair germination and/or promoting hair generation and hair growth, muscular dystrophy, pyoderma and Sezary's syndrome, Addison's disease, ischemia-reperfusion injury of organs which occurs upon preservation, transplantation or ischemic disease, endotoxin-shock, pseudomembranous colitis, colitis caused by drug or radiation, ischemic acute renal insufficiency, chronic renal insufficiency, toxinosis caused by lung-oxygen or drugs, lung cancer, pulmonary emphysema, cataracta, siderosis, retinitis pigmentosa, senile macular degeneration, vitreal scarring, corneal alkali burn, dermatitis erythema multiforme, linear IgA ballous dermatitis and cement dermatitis, gingivitis, periodontitis, sepsis, pancreatitis, diseases caused by environmental pollution, aging, carcinogenesis, metastasis of carcinoma and hypobaropathy, disease caused by histamine or leukotriene-C₄ release, Behcet's disease, autoimmune hepatitis, primary biliary cirrhosis, sclerosing cholangitis, partial liver resection, acute liver necrosis, necrosis caused by toxin, viral hepatitis, shock, or anoxia, B-virus hepatitis, non-A/non-B hepatitis, cirrhosis, alcoholic cirrhosis, hepatic failure, fulminant hepatic failure, late-onset hepatic failure, “acute-on-chronic” liver failure, augmentation of chemotherapeutic effect, cytomegalovirus infection, HCMV infection, AIDS, cancer, senile dementia, trauma, and chronic bacterial infection.

The method may further comprise administering an effective amount of one or more other agents to the subject before, during, and/or after administration of the compound of the invention. For example, in the case of cancer, an anti-cancer agent can be administered.

By way of example, peptoid bodies that target malaria-infected erythrocytes for immune attack by host macrophages may be identified. A differential cell-binding screen can be used to determine which members of the peptoid body library will recognize Plasmodium falciparum infected human erythrocytes. The hits that bind only the infected cells will have presumably bound to an extracellular target that is only expressed on or that is overexpressed on the infected cells as compared with the uninfected cells. The hit is resynthesized to verify its specific binding to the infected cells vs. the uninfected cells, followed by a resynthesis to contain biotin. Standard pull down procedures can be used to isolate the proteins either directly binding the hit or proteins associated with the direct binding partner. The isolated proteins will be subject to proteomics analysis. Photoaffinity labeling of the proteins can be performed under denaturing conditions to dissociate noncovalent protein-protein aggregates to rigorously determine the direct hit molecule-binding partner using proteomic analysis. If this target is a known and readily available recombinant protein, direct binding of the hit with the target can be verified in vitro using a surface plasmon resonance-binding assay. The peptoid-body library can be screened for binding to IFN gamma that has been fluorescently labeled. A tight-binding IFN gamma ligand that does not block the normal IFN gamma binding with the IFN gamma receptor found on macrophages is desirable. The small size of peptoid-body ligands and the easy specific conjugation of peptoid-body ligands will make the heterodimerization of a targeting ligand for P. falciparum infected human erythrocytes with a IFN gamma ligand much easier than it would be using two different antibodies to bring these normally disparate components together. This approach of specifically targeting macrophages to P. falciparum infected human erythrocytes will involve titration of a dosage that does not cause a septic shock from an overwhelming immune response from this purposeful macrophage onslaught on the targeted erythrocytes. The identification of specific targeting ligands for extracellular targets associated with malaria infection can provide valuable targeted therapeutic agents.

Another aspect of the inventions concerns a method for inducing apoptosis or inhibiting the growth of a cell, comprising contacting the cell with an effective amount of a peptoid body in vitro or in vivo. In some embodiments, the cell is a cancer cell. In some embodiments, the cell is a pathogen-infected cell (e.g., a malaria-infected cell). The method may further comprise contacting the cell with one or more other agents before, during, and/or after contacting the cell with the peptoid body. For example, in the case of a cancer cell, the cell may be contacted with one or more anti-cancer agents.

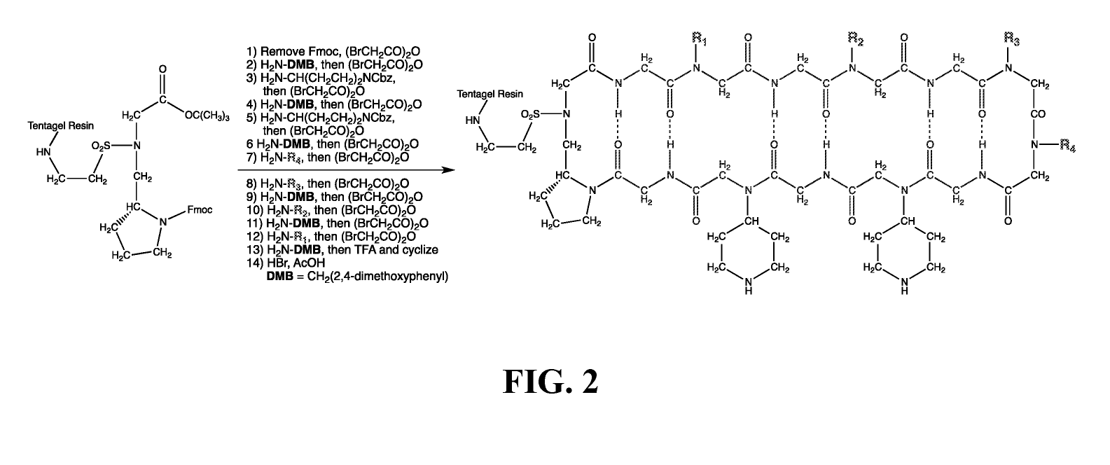

An exemplary preparation of a substrate-bound (e.g., resin-bound) peptoid body is shown in FIG. 2. In an embodiment of the invention, a method for the preparation of a resin-bound peptoid body involves deprotecting an amine of a resin-bound beta turn promoter by the removal of an Fmoc group followed by an amide formation by reaction with dibromoacetic anhydride. Subsequent nucleophilic substitution with 2,4-dimethoxybenzylamine (H₂NDMB) generates a mono-N-protected glycine unit on the end of the growing peptoid body. The reactions with bromoacetic anhydride and a primary amine, either a primary amine with a desired R group or H₂NDMB is repeated until all of the desired amino acid residues of the desired peptoid body are added. Ultimately, the steps of cyclization and deprotection result in the cyclic peptoid-peptide hybrid bound to the substrate.

Compounds of the subject invention also include pharmaceutically acceptable salts and hydrates of the subject compounds. Pharmaceutically acceptable salts include salts of the compounds of the invention, which are prepared with acids or bases, depending on the particular substituents found on the subject complexes described herein. Examples of pharmaceutically acceptable base addition salts include sodium, potassium, calcium, ammonium, or magnesium salt. Examples of pharmaceutically acceptable acid addition salts include hydrochloric, hydrobromic, nitric, phosphoric, carbonic, sulphuric, and organic acids like acetic, propionic, benzoic, succinic, fumaric, mandelic, oxalic, citric, tartaric, maleic, and the like. Pharmaceutically acceptable salts of compounds of the invention can be prepared using conventional techniques.

It will be appreciated by those skilled in the art that some of the compounds of the invention may contain one or more asymmetrically substituted carbon atoms, which can give rise to stereoisomers. It is understood that the invention extends to all such stereoisomers, including enantiomers, and diastereoisomers and mixtures, including racemic mixtures thereof.

The subject invention also concerns a packaged dosage formulation comprising in one or more containers at least one peptoid-peptide hybrid or composition of the invention. A packaged dosage formulation can optionally comprise in one or more containers a pharmaceutically acceptable carrier or diluent. A packaged dosage formulation can also optionally comprise, in addition to peptoid body or a composition of the invention, other agents such as anti-cancer agents, including, but not limited to, chemotherapeutic drugs.

In vivo application of the peptoid-peptide hybrids of the invention, and compositions containing them, can be accomplished by any suitable method and technique presently or prospectively known to those skilled in the art. The subject compounds can be formulated in a pharmaceutically acceptable form and administered by any suitable route known in the art including, for example, oral, nasal, rectal, and parenteral routes of administration. As used herein, the term parenteral includes subcutaneous, intradermal, intravenous, intramuscular, intraperitoneal, and intrasternal administration, such as by injection. Administration of the subject PPIIs of the invention can be a single administration, or at continuous or distinct intervals as can be readily determined by a person skilled in the art.

The peptoid-peptide hybrids of the subject invention, and compositions comprising them, can also be administered utilizing liposome technology, slow release capsules, implantable pumps, and biodegradable containers. These delivery methods can, advantageously, provide a uniform dosage over an extended period. The compounds of the invention can also be administered in their salt derivative forms or crystalline forms.

PPIIs of the subject invention can be formulated according to known methods for preparing physiologically acceptable compositions. Formulations are described in detail in a number of sources, which are well known and readily available to those skilled in the art. For example, Remington's Pharmaceutical Science by E. W. Martin describes formulations, which can be used in connection with the subject invention. In general, the compositions of the subject invention will be formulated such that an effective amount of the compound is combined with a suitable carrier in order to facilitate effective administration of the composition. The compositions used in the present methods can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional physiologically-acceptable carriers and diluents, which are known to those skilled in the art. Examples of carriers or diluents for use with the subject compounds include ethanol, dimethyl sulfoxide, glycerol, alumina, starch, saline, and equivalent carriers and diluents. To provide for the administration of such dosages for the desired therapeutic treatment, compositions of the invention will advantageously comprise between about 0.1% and 99%, and especially, 1 and 15% by weight of the total of one or more of the subject compounds based on the weight of the total composition including carrier or diluent.

Peptoid-peptide hybrids of the invention, and compositions comprising them, can be delivered to a cell (e.g., normal cell, cancerous cell, cell line) either through direct contact with the cell or via a carrier means. In some embodiments, the cell to which the compound or composition is contacted is a cancer cell of a type disclosed herein (e.g., Table 2). Carrier means for delivering compounds and compositions to cells are known in the art and include, for example, encapsulating the composition in a liposome moiety. Another means for delivery of compounds and compositions of the invention to a cell comprises attaching the compounds to a protein or nucleic acid that is targeted for delivery to the target cell. U.S. Pat. No. 6,960,648 and Published U.S. Patent Application Nos. 20030032594 and 20020120100 disclose amino acid sequences that can be coupled to another composition and that allows the composition to be translocated across biological membranes. Published U.S. Patent Application No. 20020035243 also describes compositions for transporting biological moieties across cell membranes for intracellular delivery. Compounds can also be incorporated into polymers, examples of which include poly(D-L lactide-co-glycolide) polymer for intracranial tumors; poly[bis(p-carboxyphenoxy) propane:sebacic acid] in a 20:80 molar ratio (as used in GLIADEL); chondroitin; chitin; and chitosan.

Oncological disorders within the scope of the invention include, but are not limited to, cancer and/or tumors of the anus, bile duct, bladder, bone, bone marrow, bowel (including colon and rectum), breast, eye, gall bladder, kidney, mouth, larynx, esophagus, stomach, testis, cervix, head, neck, ovary, lung, mesothelioma, neuroendocrine, penis, skin, spinal cord, thyroid, vagina, vulva, uterus, liver, muscle, pancreas, prostate, blood cells (including lymphocytes and other immune system cells), and brain. Specific cancers contemplated for treatment with the present invention include carcinomas, Karposi's sarcoma, melanoma, mesothelioma, soft tissue sarcoma, pancreatic cancer, lung cancer, leukemia (acute lymphoblastic, acute myeloid, chronic lymphocytic, chronic myeloid, and other), and lymphoma (Hodgkin's and non-Hodgkin's), and multiple myeloma.

Examples of cancers that can be treated according to the present invention are listed in Table 2.

TABLE 2
Examples of Cancer Types

| | |
|---|---|
| Acute Lymphoblastic Leukemia, Adult | Hairy Cell Leukemia |
| Acute Lymphoblastic Leukemia, | Head and Neck Cancer |
| Childhood | Hepatocellular (Liver) Cancer, Adult |
| Acute Myeloid Leukemia, Adult | (Primary) |
| Acute Myeloid Leukemia, Childhood | Hepatocellular (Liver) Cancer, Childhood |
| Adrenocortical Carcinoma | (Primary) |
| Adrenocortical Carcinoma, Childhood | Hodgkin's Lymphoma, Adult |
| AIDS-Related Cancers | Hodgkin's Lymphoma, Childhood |
| AIDS-Related Lymphoma | Hodgkin's Lymphoma During Pregnancy |
| Anal Cancer | Hypopharyngeal Cancer |
| Astrocytoma, Childhood Cerebellar | Hypothalamic and Visual Pathway Glioma, |
| Astrocytoma, Childhood Cerebral | Childhood |
| Basal Cell Carcinoma | Intraocular Melanoma |
| Bile Duct Cancer, Extrahepatic | Islet Cell Carcinoma (Endocrine Pancreas) |
| Bladder Cancer | Kaposi's Sarcoma |
| Bladder Cancer, Childhood | Kidney (Renal Cell) Cancer |
| Bone Cancer, Osteosarcoma/Malignant | Kidney Cancer, Childhood |
| Fibrous Histiocytoma | Laryngeal Cancer |
| Brain Stem Glioma, Childhood | Laryngeal Cancer, Childhood |
| Brain Tumor, Adult | Leukemia, Acute Lymphoblastic, Adult |
| Brain Tumor, Brain Stem Glioma, | Leukemia, Acute Lymphoblastic, Childhood |
| Childhood | Leukemia, Acute Myeloid, Adult |
| Brain Tumor, Cerebellar Astrocytoma, | Leukemia, Acute Myeloid, Childhood |
| Childhood | Leukemia, Chronic Lymphocytic |
| Brain Tumor, Cerebral | Leukemia, Chronic Myelogenous |
| Astrocytoma/Malignant Glioma, | Leukemia, Hairy Cell |
| Childhood | Lip and Oral Cavity Cancer |
| Brain Tumor, Ependymoma, Childhood | Liver Cancer, Adult (Primary) |
| Brain Tumor, Medulloblastoma, | Liver Cancer, Childhood (Primary) |
| Childhood | Lung Cancer, Non-Small Cell |
| Brain Tumor, Supratentorial Primitive | Lung Cancer, Small Cell |
| Neuroectodermal Tumors, Childhood | Lymphoma, AIDS-Related |
| Brain Tumor, Visual Pathway and | Lymphoma, Burkitt's |
| Hypothalamic Glioma, Childhood | Lymphoma, Cutaneous T-Cell, see Mycosis |
| Brain Tumor, Childhood | Fungoides and Sézary Syndrome |
| Breast Cancer | Lymphoma, Hodgkin's, Adult |
| Breast Cancer, Childhood | Lymphoma, Hodgkin's, Childhood |
| Breast Cancer, Male | Lymphoma, Hodgkin's During Pregnancy |
| Bronchial Adenomas/Carcinoids, | Lymphoma, Non-Hodgkin's, Adult |
| Childhood | Lymphoma, Non-Hodgkin's, Childhood |
| Burkitt's Lymphoma | Lymphoma, Non-Hodgkin's During |
| Carcinoid Tumor, Childhood | Pregnancy |
| Carcinoid Tumor, Gastrointestinal | Lymphoma, Primary Central Nervous System |
| Carcinoma of Unknown Primary | Macroglobulinemia, Waldenström's |
| Central Nervous System Lymphoma, | Malignant Fibrous Histiocytoma of |
| Primary | Bone/Osteosarcoma |
| Cerebellar Astrocytoma, Childhood | Medulloblastoma, Childhood |
| Cerebral Astrocytoma/Malignant | Melanoma |
| Glioma, Childhood | Melanoma, Intraocular (Eye) |
| Cervical Cancer | Merkel Cell Carcinoma |
| Childhood Cancers | Mesothelioma, Adult Malignant |
| Chronic Lymphocytic Leukemia | Mesothelioma, Childhood |
| Chronic Myelogenous Leukemia | Metastatic Squamous Neck Cancer with |
| Chronic Myeloproliferative Disorders | Occult Primary |
| Colon Cancer | Multiple Endocrine Neoplasia Syndrome, |
| Colorectal Cancer, Childhood | Childhood |
| Cutaneous T-Cell Lymphoma, see | Multiple Myeloma/Plasma Cell Neoplasm |
| Mycosis Fungoides and Sezary | Mycosis Fungoides |
| Syndrome | Myelodysplastic Syndromes |
| Endometrial Cancer | Myelodysplastic/Myeloproliferative Diseases |
| Ependymoma, Childhood | Myelogenous Leukemia, Chronic |
| Esophageal Cancer | Myeloid Leukemia, Adult Acute |
| Esophageal Cancer, Childhood | Myeloid Leukemia, Childhood Acute |
| Ewing's Family of Tumors | Myeloma, Multiple |
| Extracranial Germ Cell Tumor, | Myeloproliferative Disorders, Chronic |
| Childhood | Nasal Cavity and Paranasal Sinus Cancer |
| Extragonadal Germ Cell Tumor | Nasopharyngeal Cancer |
| Extrahepatic Bile Duct Cancer | Nasopharyngeal Cancer, Childhood |
| Eye Cancer, Intraocular Melanoma | Neuroblastoma |
| Eye Cancer, Retinoblastoma | Non-Hodgkin's Lymphoma, Adult |
| Gallbladder Cancer | Non-Hodgkin's Lymphoma, Childhood |
| Gastric (Stomach) Cancer | Non-Hodgkin's Lymphoma During Pregnancy |
| Gastric (Stomach) Cancer, Childhood | Non-Small Cell Lung Cancer |
| Gastrointestinal Carcinoid Tumor | Oral Cancer, Childhood |
| Germ Cell Tumor, Extracranial, | Oral Cavity Cancer, Lip and |
| Childhood | Oropharyngeal Cancer |
| Germ Cell Tumor, Extragonadal | Osteosarcoma/Malignant Fibrous |
| Germ Cell Tumor, Ovarian | Histiocytoma of Bone |
| Gestational Trophoblastic Tumor | Ovarian Cancer, Childhood |
| Glioma, Adult | Ovarian Epithelial Cancer |
| Glioma, Childhood Brain Stem | Ovarian Germ Cell Tumor |
| Glioma, Childhood Cerebral | Ovarian Low Malignant Potential Tumor |
| Astrocytoma | Pancreatic Cancer |
| Glioma, Childhood Visual Pathway and | Pancreatic Cancer, Childhood |
| Hypothalamic | Pancreatic Cancer, Islet Cell |
| Skin Cancer (Melanoma) | Paranasal Sinus and Nasal Cavity Cancer |
| Skin Carcinoma, Merkel Cell | Parathyroid Cancer |
| Small Cell Lung Cancer | Penile Cancer |
| Small Intestine Cancer | Pheochromocytoma |
| Soft Tissue Sarcoma, Adult | Pineoblastoma and Supratentorial Primitive |
| Soft Tissue Sarcoma, Childhood | Neuroectodermal Tumors, Childhood |
| Squamous Cell Carcinoma, see Skin | Pituitary Tumor |
| Cancer (non-Melanoma) | Plasma Cell Neoplasm/Multiple Myeloma |
| Squamous Neck Cancer with Occult | Pleuropulmonary Blastoma |
| Primary, Metastatic | Pregnancy and Breast Cancer |
| Stomach (Gastric) Cancer | Pregnancy and Hodgkin's Lymphoma |
| Stomach (Gastric) Cancer, Childhood | Pregnancy and Non-Hodgkin's Lymphoma |
| Supratentorial Primitive | Primary Central Nervous System Lymphoma |
| Neuroectodermal Tumors, Childhood | Prostate Cancer |
| T-Cell Lymphoma, Cutaneous, see | Rectal Cancer |
| Mycosis Fungoides and Sezary | Renal Cell (Kidney) Cancer |
| Syndrome | Renal Cell (Kidney) Cancer, Childhood |
| Testicular Cancer | Renal Pelvis and Ureter, Transitional Cell |
| Thymoma, Childhood | Cancer |
| Thymoma and Thymic Carcinoma | Retinoblastoma |
| Thyroid Cancer | Rhabdomyosarcoma, Childhood |
| Thyroid Cancer, Childhood | Salivary Gland Cancer |
| Transitional Cell Cancer of the Renal | Salivary Gland Cancer, Childhood |
| Pelvis and Ureter | Sarcoma, Ewing's Family of Tumors |
| Trophoblastic Tumor, Gestational | Sarcoma, Kaposi's |
| Unknown Primary Site, Carcinoma of, | Sarcoma, Soft Tissue, Adult |
| Adult | Sarcoma, Soft Tissue, Childhood |
| Unknown Primary Site, Cancer of, | Sarcoma, Uterine |
| Childhood | Sezary Syndrome |
| Unusual Cancers of Childhood | Skin Cancer (non-Melanoma) |
| Ureter and Renal Pelvis, Transitional | Skin Cancer, Childhood |
| Cell Cancer | |
| Urethral Cancer | |
| Uterine Cancer, Endometrial | |
| Uterine Sarcoma | |
| Vaginal Cancer | |
| Visual Pathway and Hypothalamic | |
| Glioma, Childhood | |
| Vulvar Cancer | |
| Waldenström's Macroglobulinemia | |
| Wilms' Tumor | |

For the treatment of oncological disorders, the PPIIs of this invention can be administered to a subject in need of treatment in combination with other anti-cancer substances and/or with anti-cancer treatments (such as radiation, photodynamic therapy, and surgical treatment) to remove a tumor. These other substances or treatments may be given at the same time as, or at different times from, the compounds and compositions of this invention. For example, the compounds of the present invention can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, anti-angiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively.

As used herein, the term “tumor” refers to all neoplastic cell growth and proliferation, whether malignant or benign, and all pre-cancerous and cancerous cells and tissues. For example, a particular cancer may be characterized by a solid mass tumor. The solid tumor mass, if present, may be a primary tumor mass. A primary tumor mass refers to a growth of cancer cells in a tissue resulting from the transformation of a normal cell of that tissue. In most cases, the primary tumor mass is identified by the presence of a cyst, which can be found through visual or palpation methods, or by irregularity in shape, texture or weight of the tissue. However, some primary tumors are not palpable and can be detected only through medical imaging techniques such as X-rays (e.g., mammography), or by needle aspirations. The use of these latter techniques is more common in early detection. Molecular and phenotypic analysis of cancer cells within a tissue will usually confirm if the cancer is endogenous to the tissue or if the lesion is due to metastasis from another site. The tumor “tumor” is inclusive of solid tumors and non-solid tumors. The PPII and compositions of the invention can be administered locally at the site of a tumor (e.g., by direct injection) or remotely.

As used herein, the terms “treat” or “treatment” refer to both therapeutic treatment and prophylactic or preventative measures, wherein the object is to prevent or slow down (lessen) an undesired physiological change or disorder, such as the development or spread of an oncological disorder (e.g., cancer), infection by a pathogenic microorganism (e.g., malaria), or immunoregulatory abnormality such as an autoimmune disease or chronic inflammatory disease. Thus, in some embodiments, the subject has an oncological disorder at the time of administration. In other embodiments, the subject does not have an oncological disorder at the time of administration, in which case the PPII may be administered to prevent or delay onset of the oncologic disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented. In some embodiments, the treatment methods include identifying the subject as having an oncologic disorder (e.g., cancer).

The amount of PPII administered to the subject may be an effective amount, e.g., a therapeutically effective amount. As used herein, the term “(therapeutically) effective amount” refers to an amount of an agent (e.g., a compound of the invention or other anti-cancer agent) effective to treat a disease or disorder in a mammal. In the case of cancer, the therapeutically effective amount of the agent may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve, to some extent, one or more of the symptoms associated with the cancer. To the extent the agent may prevent growth and/or kill existing cancer cells, it may be cytostatic and/or cytotoxic. For cancer therapy, efficacy can, for example, be measured by assessing the time to disease progression (TTP) and/or determining the response rate (RR).

The amount of PPII administered to the subject may be a growth inhibitory amount. As used herein, the term “growth inhibitory amount” refers to an amount which inhibits growth of a target cell, such as a tumor cell, either in vitro or in vivo, irrespective of the mechanism by which cell growth is inhibited. In a preferred embodiment, the growth inhibitory amount inhibits growth of the target cell in cell culture by greater than about 20%, preferably greater than about 50%, most preferably greater than about 75% (e.g., from about 75% to about 100%).

The terms “cell” and “cells” are used interchangeably herein and are intended to include either a single cell or a plurality of cells unless otherwise specified.

As used herein, the term “anti-cancer agent” refers to a substance or treatment that inhibits the function of cancer cells, inhibits their formation, and/or causes their destruction in vitro or in vivo. Examples include, but are not limited to, cytotoxic agents (e.g., 5-fluorouracil, TAXOL) and anti-signaling agents (e.g., the PI3K inhibitor LY).

The methods of the present invention can be used with humans and other animal subjects. The other animals contemplated within the scope of the invention include domesticated, agricultural, or zoo- or circus-maintained animals. Domesticated animals include, for example, dogs, cats, rabbits, ferrets, guinea pigs, hamsters, pigs, monkeys or other primates, and gerbils. Agricultural animals include, for example, horses, mules, donkeys, burros, cattle, cows, pigs, sheep, and alligators. Zoo- or circus-maintained animals include, for example, lions, tigers, bears, camels, giraffes, hippopotamuses, and rhinoceroses. In one embodiment, the subject is a human or non-human mammal.

While PPIIs can be administered as isolated compounds, these compounds can also be administered as part of a pharmaceutical composition. The subject invention thus further provides compositions comprising one or more PPIIs in association with at least one pharmaceutically acceptable carrier or diluent. The pharmaceutical composition can be adapted for various routes of administration, such as enteral, parenteral, intravenous, intramuscular, topical, subcutaneous, and so forth. Administration can be continuous or at distinct intervals, as can be determined by a person of ordinary skill in the art.

The PPIIs can be formulated according to known methods for preparing pharmaceutically useful compositions. Formulations are described in a number of sources which are well known and readily available to those skilled in the art. For example, Remington's Pharmaceutical Science (Martin 1995) describes formulations which can be used in connection with the subject invention. Formulations suitable for administration include, for example, aqueous sterile injection solutions, which may contain antioxidants, buffers, bacteriostats, and solutes that render the formulation isotonic with the blood of the intended recipient; and aqueous and nonaqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the compositions of the subject invention can include other agents conventional in the art having regard to the type of formulation in question.

The PPIIs of the present invention include all hydrates and salts that can be prepared by those of skill in the art. Under conditions where the compounds and agents of the present invention are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, alpha-ketoglutarate, and alpha-glycerophosphate. Suitable inorganic salts may also be formed, including hydrochloride, sulfate, nitrate, bicarbonate, and carbonate salts.

Pharmaceutically acceptable salts of a compound or agent may be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Therapeutic application of PPIIs and compositions containing them can be accomplished by any suitable therapeutic method and technique presently or prospectively known to those skilled in the art. Further, compounds and agents of the invention have use as starting materials or intermediates for the preparation of other useful compounds and compositions.

PPIIs of the invention, and compositions thereof, may be locally administered at one or more anatomical sites, such as sites of unwanted cell growth (such as a tumor site or benign skin growth, e.g., injected or topically applied to the tumor or skin growth), sites of infection or other sites of diseased cells, optionally in combination with a pharmaceutically acceptable carrier such as an inert diluent. Compounds and agents of the invention, and compositions thereof, may be systemically administered, such as intravenously or orally, optionally in combination with a pharmaceutically acceptable carrier such as an inert diluent, or an assimilable edible carrier for oral delivery. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, aerosol sprays, and the like.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or as otherwise modifications of the physical form of the solid unit dosage form. For instance, tablets, pills, or capsules may be coated with gelatin, wax, shellac, or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

PPIIs and compositions of the invention, including pharmaceutically acceptable salts or hydrates thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating one or more PPIIs of the invention (with or without one or more additional anti-cancer agents) in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, PPIIs and compositions of the invention may be applied in as a liquid or solid. However, it will generally be desirable to administer them topically to the skin as compositions, in combination with a dermatologically acceptable carrier, which may be a solid or a liquid. Compounds and agents and compositions of the subject invention can be applied topically to a subject's skin to reduce the size (and may include complete removal) of malignant or benign growths, or to treat an infection site. Compounds and agents of the invention can be applied directly to the diseased site (e.g., the growth or infection site). Preferably, the compounds and agents are applied to the growth or infection site in a formulation such as an ointment, cream, lotion, solution, tincture, or the like. Drug delivery systems for delivery of pharmacological substances to dermal lesions can also be used, such as that described in U.S. Pat. No. 5,167,649.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user. Examples of useful dermatological compositions which can be used to deliver a compound to the skin are disclosed in U.S. Pat. No. 4,608,392; U.S. Pat. No. 4,992,478; U.S. Pat. No. 4,559,157; and U.S. Pat. No. 4,820,508.

Useful dosages of the PPIIs pharmaceutical compositions of the present invention can be determined by comparing their in vitro activity, and in vivo activity in animal models. Methods for the extrapolation of effective dosages in mice, and other animals, to humans are known to the art; for example, see U.S. Pat. No. 4,938,949.

The present invention also concerns pharmaceutical compositions comprising one or more PPIIs of the invention in combination with a pharmaceutically acceptable carrier. Pharmaceutical compositions adapted for oral, topical or parenteral administration, comprising an amount of a compound constitute a preferred embodiment of the invention. The dose administered to a patient, particularly a human, in the context of the present invention should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

For the treatment of oncological disorders, PPIIs and compositions containing them contemplated by the present invention can be administered to a subject in need of treatment prior to, subsequent to, or in combination with other anti-cancer agents or treatments (e.g., chemotherapeutic agents, immunotherapeutic agents, radiotherapeutic agents, cytotoxic agents, etc.) and/or with radiation therapy and/or with surgical treatment to remove a tumor. For example, compounds and compositions of the present invention can be used in methods of treating cancer wherein the subject is to be treated or is or has been treated with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively. These other substances or radiation treatments may be given at the same as or at different times from the compounds of this invention. Examples of other chemotherapeutic agents contemplated within the scope of the invention include, but are not limited to, altretamine, bleomycin, bortezomib (VELCADE), busulphan, calcium folinate, capecitabine, carboplatin, carmustine, chlorambucil, cisplatin, cladribine, crisantaspase, cyclophosphamide, cytarabine, dacarbazine, dactinomycin, daunorubicin, docetaxel, doxorubicin, epirubicin, etoposide, fludarabine, fluorouracil, gefitinib (IRESSA), gemcitabine, hydroxyurea, idarubicin, ifosfamide, imatinib (GLEEVEC), irinotecan, liposomal doxorubicin, lomustine, melphalan, mercaptopurine, methotrexate, mitomycin, mitoxantrone, oxaliplatin, paclitaxel, pentostatin, procarbazine, raltitrexed, streptozocin, tegafur-uracil, temozolomide, thiotepa, tioguanine/thioguanine, topotecan, treosulfan, vinblastine, vincristine, vindesine, vinorelbine. In an exemplified embodiment, the chemotherapeutic agent is melphalan. Examples of immunotherapeutic agents contemplated within the scope of the invention include, but are not limited to, alemtuzumab, cetuximab (ERBITUX), gemtuzumab, iodine 131 tositumomab, rituximab, trastuzamab (HERCEPTIN). Cytotoxic agents include, for example, radioactive isotopes (e.g., I¹³¹, I¹²⁵, Y⁹⁰, P³², etc.), and toxins of bacterial, fungal, plant, or animal origin (e.g., ricin, botulinum toxin, anthrax toxin, aflatoxin, jellyfish venoms (e.g., box jellyfish), etc.) The subject invention also concerns methods for treating an oncological disorder comprising administering an effective amount of a compound and/or agent of the invention prior to, subsequent to, and/or in combination with administration of a chemotherapeutic agent, an immunotherapeutic agent, a radiotherapeutic agent, or radiotherapy.

Examples of some chemotherapeutic agents that can be used according to the present invention are listed in Table 3.

TABLE 3
Examples of Chemotherapeutic Agents

| | |
|---|---|
| 13-cis-Retinoic Acid | Mylocel |
| 2-Amino-6- | Letrozole |
| Mercaptopurine | Neosar |
| 2-CdA | Neulasta |
| 2-Chlorodeoxyadenosine | Neumega |
| 5-fluorouracil | Neupogen |
| 5-FU | Nilandron |
| 6-TG | Nilutamide |
| 6-Thioguanine | Nitrogen Mustard |
| 6-Mercaptopurine | Novaldex |
| 6-MP | Novantrone |
| Accutane | Octreotide |
| Actinomycin-D | Octreotide acetate |
| Adriamycin | Oncospar |
| Adrucil | Oncovin |
| Agrylin | Ontak |
| Ala-Cort | Onxal |
| Aldesleukin | Oprevelkin |
| Alemtuzumab | Orapred |
| Alitretinoin | Orasone |
| Alkaban-AQ | Oxaliplatin |
| Alkeran | Paclitaxel |
| All-transretinoic acid | Pamidronate |
| Alpha interferon | Panretin |
| Altretamine | Paraplatin |
| Amethopterin | Pediapred |
| Amifostine | PEG Interferon |
| Aminoglutethimide | Pegaspargase |
| Anagrelide | Pegfilgrastim |
| Anandron | PEG-INTRON |
| Anastrozole | PEG-L-asparaginase |
| Arabinosylcytosine | Phenylalanine Mustard |
| Ara-C | Platinol |
| Aranesp | Platinol-AQ |
| Aredia | Prednisolone |
| Arimidex | Prednisone |
| Aromasin | Prelone |
| Arsenic trioxide | Procarbazine |
| Asparaginase | PROCRIT |
| ATRA | Proleukin |
| Avastin | Prolifeprospan 20 with Carmustine implant |
| BCG | Purinethol |
| BCNU | Raloxifene |
| Bevacizumab | Rheumatrex |
| Bexarotene | Rituxan |
| Bicalutamide | Rituximab |
| BiCNU | Roveron-A (interferon alfa-2a) |
| Blenoxane | Rubex |
| Bleomycin | Rubidomycin hydrochloride |
| Bortezomib | Sandostatin |
| Busulfan | Sandostatin LAR |
| Busulfex | Sargramostim |
| C225 | Solu-Cortef |
| Calcium Leucovorin | Solu-Medrol |
| Campath | STI-571 |
| Camptosar | Streptozocin |
| Camptothecin-11 | Tamoxifen |
| Capecitabine | Targretin |
| Carac | Taxol |
| Carboplatin | Taxotere |
| Carmustine | Temodar |
| Carmustine wafer | Temozolomide |
| Casodex | Teniposide |
| CCNU | TESPA |
| CDDP | Thalidomide |
| CeeNU | Thalomid |
| Cerubidine | TheraCys |
| cetuximab | Thioguanine |
| Chlorambucil | Thioguanine Tabloid |
| Cisplatin | Thiophosphoamide |
| Citrovorum Factor | Thioplex |
| Cladribine | Thiotepa |
| Cortisone | TICE |
| Cosmegen | Toposar |
| CPT-11 | Topotecan |
| Cyclophosphamide | Toremifene |
| Cytadren | Trastuzumab |
| Cytarabine | Tretinoin |
| Cytarabine liposomal | Trexall |
| Cytosar-U | Trisenox |
| Cytoxan | TSPA |
| Dacarbazine | VCR |
| Dactinomycin | Velban |
| Darbepoetin alfa | Velcade |
| Daunomycin | VePesid |
| Daunorubicin | Vesanoid |
| Daunorubicin | Viadur |
| hydrochloride | Vinblastine |
| Daunorubicin liposomal | Vinblastine Sulfate |
| DaunoXome | Vincasar Pfs |
| Decadron | Vincristine |
| Delta-Cortef | Vinorelbine |
| Deltasone | Vinorelbine tartrate |
| Denileukin diftitox | VLB |
| DepoCyt | VP-16 |
| Dexamethasone | Vumon |
| Dexamethasone acetate | Xeloda |
| dexamethasone sodium | Zanosar |
| phosphate | Zevalin |
| Dexasone | Zinecard |
| Dexrazoxane | Zoladex |
| DHAD | Zoledronic acid |
| DIC | Zometa |
| Diodex | Gliadel wafer |
| Docetaxel | Glivec |
| Doxil | GM-CSF |
| Doxorubicin | Goserelin |
| Doxorubicin liposomal | granulocyte - colony stimulating factor |
| Droxia | Granulocyte macrophage colony stimulating |
| DTIC | factor |
| DTIC-Dome | Halotestin |
| Duralone | Herceptin |
| Efudex | Hexadrol |
| Eligard | Hexalen |
| Ellence | Hexamethylmelamine |
| Eloxatin | HMM |
| Elspar | Hycamtin |
| Emcyt | Hydrea |
| Epirubicin | Hydrocort Acetate |
| Epoetin alfa | Hydrocortisone |
| Erbitux | Hydrocortisone sodium phosphate |
| Erwinia L-asparaginase | Hydrocortisone sodium succinate |
| Estramustine | Hydrocortone phosphate |
| Ethyol | Hydroxyurea |
| Etopophos | Ibritumomab |
| Etoposide | Ibritumomab Tiuxetan |
| Etoposide phosphate | Idamycin |
| Eulexin | Idarubicin |
| Evista | Ifex |
| Exemestane | IFN-alpha |
| Fareston | Ifosfamide |
| Faslodex | IL-2 |
| Femara | IL-11 |
| Filgrastim | Imatinib mesylate |
| Floxuridine | Imidazole Carboxamide |
| Fludara | Interferon alfa |
| Fludarabine | Interferon Alfa-2b (PEG conjugate) |
| Fluoroplex | Interleukin-2 |
| Fluorouracil | Interleukin-11 |
| Fluorouracil (cream) | Intron A (interferon alfa-2b) |
| Fluoxymesterone | Leucovorin |
| Flutamide | Leukeran |
| Folinic Acid | Leukine |
| FUDR | Leuprolide |
| Fulvestrant | Leurocristine |
| G-CSF | Leustatin |
| Gefitinib | Liposomal Ara-C |
| Gemcitabine | Liquid Pred |
| Gemtuzumab ozogamicin | Lomustine |
| Gemzar | L-PAM |
| Gleevec | L-Sarcolysin |
| Lupron | Meticorten |
| Lupron Depot | Mitomycin |
| Matulane | Mitomycin-C |
| Maxidex | Mitoxantrone |
| Mechlorethamine | M-Prednisol |
| Mechlorethamine | MTC |
| Hydrochlorine | MTX |
| Medralone | Mustargen |
| Medrol | Mustine |
| Megace | Mutamycin |
| Megestrol | Myleran |
| Megestrol Acetate | Iressa |
| Melphalan | Irinotecan |
| Mercaptopurine | Isotretinoin |
| Mesna | Kidrolase |
| Mesnex | Lanacort |
| Methotrexate | L-asparaginase |
| Methotrexate Sodium | LCR |
| Methylprednisolone | |

Depending upon the disorder or disease condition to be treated, a suitable dose(s) may be that amount that will reduce proliferation or growth of the target cell(s). In the context of cancer, a suitable dose(s) is that which will result in a concentration of the active agent in cancer tissue, such as a malignant tumor, which is known to achieve the desired response. The preferred dosage is the amount which results in maximum inhibition of cancer cell growth, without unmanageable side effects. Administration of a compound and/or agent can be continuous or at distinct intervals, as can be determined by a person of ordinary skill in the art.

To provide for the administration of such dosages for the desired therapeutic treatment, in some embodiments, pharmaceutical compositions of the invention can comprise between about 0.1% and 45%, and especially, 1 and 15%, by weight of the total of one or more of the compounds based on the weight of the total composition including carrier or diluents. Illustratively, dosage levels of the administered active ingredients can be: intravenous, 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, and preferably about 1 to 100 mg/kg; intranasal instillation, 0.01 to about 20 mg/kg; and aerosol, 0.01 to about 20 mg/kg of animal (body) weight. The subject invention also concerns kits comprising a composition comprising PPII or composition of the invention in one or more containers. Kits of the invention can optionally include pharmaceutically acceptable carriers and/or diluents. In one embodiment, a kit of the invention includes one or more other components, adjuncts, or adjuvants as described herein. In another embodiment, a kit includes one or more anti-cancer agents, such as those agents described herein. In one embodiment, a kit of the invention includes instructions or packaging materials that describe how to administer a compound or composition of the kit. Containers of the kit can be of any suitable material, e.g., glass, plastic, metal, etc., and of any suitable size, shape, or configuration. In one embodiment, a compound or composition of the invention is provided in the kit as a solid, such as a tablet, pill, or powder form. In another embodiment, a compound or composition of the invention is provided in the kit as a liquid or solution. In one embodiment, the kit comprises an ampoule or syringe containing a compound and/or agent of the invention in liquid or solution form. A kit of the invention can also optionally comprise, in addition to a PPII or composition of the invention, one or more other anticancer agents, such as chemotherapeutic agents.

Mammalian species which benefit from the disclosed methods include, but are not limited to, primates, such as apes, chimpanzees, orangutans, humans, monkeys; domesticated animals (e.g., pets) such as dogs, cats, guinea pigs, hamsters, Vietnamese pot-bellied pigs, rabbits, and ferrets; domesticated farm animals such as cows, buffalo, bison, horses, donkey, swine, sheep, and goats; exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, opossums, raccoons, pandas, hyena, seals, sea lions, elephant seals, otters, porpoises, dolphins, and whales. Other species that may benefit from the disclosed methods include fish, amphibians, avians, and reptiles. As used herein, the terms “patient” and “subject” are used interchangeably and are intended to include such human and non-human species. Likewise, in vitro methods of the present invention can be carried out on cells of such human and non-human species.

PPIIs as described herein may include residues of L-amino acids, D-amino acids, or any combination thereof. In some embodiments, all amino acids of the peptide are D-amino acids. Amino acids may be from natural or non-natural sources. Unless indicated to the contrary, the 20 L-amino acids commonly found in proteins are identified herein by the conventional one-letter or three-letter abbreviations known in the art, and the corresponding D-amino acids are generally designated by a lower case one letter symbol.

For convenience, the relationship of the three-letter abbreviation and the one-letter symbol for amino acids is provided in Table 4.

TABLE 4
Three Letter and One Letter Amino Acid Abbreviations

| Amino acid | Three letter | One letter |
|---|---|---|
| Alanine | Ala | A |
| Arginine | Arg | R |
| Asparagine | Asn | N |
| aspartic acid | Asp | D |
| asparagine or aspartic acid | Asx | B |
| Cysteine | Cys | C |
| glutamic acid | Glu | E |
| Glutamine | Gln | Q |
| glutamine or glutamic acid | Glx | Z |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Theonine | Thr | T |
| Tryptophan | Try | W |
| Tyrosine | Tyr | Y |
| Valine | Val | V |

PPIIs may also contain one or more rare amino acids (such as 4-hydroxyproline or hydroxylysine), organic acids or amides and/or derivatives of common amino acids, such as amino acids having the C-terminal carboxylate esterified (e.g., benzyl, methyl or ethyl ester) or amidated and/or having modifications of the N-terminal amino group (e.g., acetylation or alkoxycarbonylamino), with or without any of a wide variety of side chain modifications and/or substitutions (e.g., methylation, benzylation, t-butylation, tosylation, alkoxycarbonylamino, and the like). Such modifications and derivatives of an amino acid sequence, and others known to those of skill in the art, are herein termed “variants”. Some derivatives include amino acids having an N-acetyl group (such that the amino group that represents the N-terminus of the linear peptide is acetylated) and/or a C-terminal amide group (i.e., the carboxy terminus of the linear peptide is amidated). Residues other than common amino acids that may be present include, but are not limited to, penicillamine, tetramethylene cysteine, pentamethylene cysteine, mercaptopropionic acid, pentamethylene-mercaptopropionic acid, 2-mercaptobenzene, 2-mercaptoaniline, 2-mercaptoproline, ornithine, diaminobutyric acid, aminoadipic acid, m-aminomethylbenzoic acid, and diaminopropionic acid.

Functional fragments according to the subject invention can comprise a contiguous span of at least 4 consecutive amino acids of a recognition sequence (also referred to as the recognition portion) and/or a non-recognition sequence (also referred to as the non-recognition portion) of the PPIIs disclosed herein. Peptides fragments according to the subject invention can be any integer in length from at least 4 consecutive amino acids to 1 amino acid less than a full length peptide (e.g., 1 amino acid less than the full length peptide). Thus, in some embodiments, functional fragments may be 4, 5, 6, 7, 8, or 9 amino acids in length (e.g., a span of 4, 5, 6, 7, 8, or 9 consecutive amino acids).

Each fragment of the subject invention can also be described in terms of its N-terminal and C-terminal positions. For example, combinations of N-terminal to C-terminal fragments of 6 contiguous amino acids to 1 amino acid less than the full length peptide of are included in the present invention. Thus, a 6 consecutive amino acid fragment could occupy positions selected from the group consisting of 1-6, 2-7, 3-8, 4-9, 5-10, etc. It is noted that all ranges used to describe any embodiment of the present invention are inclusive unless specifically set forth otherwise and that fragments of a given peptide can be any integer in length, provided that the length of the peptide fragment is at least one amino acid shorter than the full-length peptide from which the fragment is derived.

Fragments, as described herein, can be obtained by cleaving the peptides of the invention with a proteolytic enzyme (such as trypsin, chymotrypsin, or collagenase) or with a chemical reagent, such as cyanogen bromide (CNBr). Alternatively, peptide fragments can be generated in a highly acidic environment, for example at pH 2.5. Such peptide fragments may be equally well prepared by chemical synthesis or using hosts transformed with an expression vector according to the invention.

In certain preferred embodiments, fragments of the peptides disclosed herein retain at least one property or activity that may be exhibited by the full-length peptide from which the fragments are derived. Thus, functional fragments of the invention may have one or more of the following properties or biological activities: 1) specifically bind to antibodies specific for the full-length peptide from which the fragment was derived; 2) specifically bind β1 integrin; 3) inhibit β1 integrin mediated cell adhesion; 4) induce ERK signaling; 5) cause apoptosis in target cells (e.g., malignant cells), by one or more mechanisms of action. Examples of assays to assess β1 integrin signaling, β1 integrin adhesion, and ERK activation are described in Gilcrease, M. S., Cancer Letters, 2007, 247 (1):1-25; Larsen M. et al., Current Opinion in Cell Biology, 2006, 18 (5):463-471; Luo B. H. and T. A. Springer, Current Opinion in Cell Biology, 2006, 18 (5):579-586.

Ligands that may find use with the PPIIs of the present invention can include but not be limited to sugars, lectins, antigens, intercalators, chelators, biotin, digoxygenin and combinations thereof. The particular choice of a dye as a labeling agent or cell uptake facilitator may depend upon physical characteristics such as absorption maxima, emission maxima, quantum yields, chemical stability and solvent solubility. A large number of fluorescent and chemiluminescent compounds have been shown to be useful for labeling proteins and nucleic acids. Examples of compounds that may be used as the dye portion can include but not be limited to xanthene, anthracene, cyanine, porphyrin and coumarin dyes. Examples of xanthene dyes that may be coupled to the peptides of the present invention can include but not be limited to fluorescein, 6-carboxyfluorescein (6-FAM), 5-carboxyfluorescein (5-Fam), 5- or 6-carboxy-4,7,2′,7′-tetrachlorofluorescein (TET), 5- or 6-carboxy-4′5′2′4′5′7′ hexachlorofluorescein (HEX), 5′ or 6′-carboxy-4′,5′-dichloro-2′,7′-dimethoxyfluorescein (JOE), 5-carboxy-2′,4′,5′,7′-tetrachlorofluorescein (ZOE) rhodol, rhodamine, tetramethylrhodamine (TAMRA), 4,7-dichlorotetramethyl rhodamine (DTAMRA), rhodamine X (ROX) and Texas Red. Examples of cyanine dyes that may find use with the peptides of the present invention can include but not be limited to Cy 3, Cy 3.5, Cy 5, Cy 5.5, Cy 7 and Cy 7.5. Other dyes that may find use with the peptides of the present invention can include but not be limited to energy transfer dyes, composite dyes and other aromatic compounds that give fluorescent signals. Chemiluminescent compounds that may be used with the peptides of the present invention can include but not be limited to dioxetane and acridinium esters. It should also be understood that ligands and dyes are not mutually exclusive groups. For instance, fluorescein is a well-known example of a moiety that has been used as a fluorescent label and also as an antigen for labeled antibodies.

The PPIIs of the invention may be monomeric or multimeric (e.g., dimers, trimers, tetramers and higher multimers). Accordingly, the present invention relates to monomers and multimers of the PPIIs of the invention, their preparation, and compositions containing them. Multimeric PPIIs of the subject invention can be derived from the same peptide sequence (“homomultimers”) or derived from different sequences disclosed herein (“heteromultimers”). A homomultimer may contain peptides having identical or different amino acid sequences; however these sequences are derived from the same original peptide. A heteromultimer refers to a multimeric peptide containing one or more heterologous peptides (i.e., peptides of different proteins) in addition to the peptides of the invention. Thus, a heteromultimer, in the context of the subject invention can refer to a multimeric peptide that contains any combination of peptides of the invention. Alternatively, a heteromultimeric peptide may comprise any peptide of the invention fused to a peptide or other element that forms a hydrophobic, hydrophilic, ionic and/or covalent association.

Multimeric peptides, as set forth herein, may be formed by hydrophobic, hydrophilic, ionic and/or covalent associations and/or may be indirectly linked, by for example, liposome formation. Thus, in one embodiment, multimers of the invention, such as, for example, homodimers or homotrimers, are formed when peptides of the invention contact one another in solution. In another embodiment, heteromultimers of the invention, such as, for example, heterotrimers or heterotetramers, are formed when peptides of the invention contact antibodies to the peptides of the invention (including antibodies to the heterologous polypeptide sequence in a fusion protein of the invention) in solution. In other embodiments, multimers of the invention are formed by covalent associations with and/or between the peptides of the invention. Examples include those peptide linkers described in U.S. Pat. No. 5,073,627 (hereby incorporated by reference).

Multimeric peptides can also be generated using chemical techniques known in the art. For example, peptides desired to be contained in the multimers of the invention may be chemically cross-linked using linker molecules and linker molecule length optimization techniques known in the art (see, e.g., U.S. Pat. No. 5,478,925, which is herein incorporated by reference in its entirety). Additionally, multimeric peptides can be generated by introducing disulfide bonds between the cysteine residues located within the sequence of the peptides that are being used to construct the multimeric polypeptide (see, e.g., U.S. Pat. No. 5,478,925, which is herein incorporated by reference in its entirety). Further, peptides of the invention may be modified by the addition of cysteine or biotin to the C terminus or N-terminus of the polypeptide and techniques known in the art may be applied to generate multimers containing one or more of these modified polypeptides (see, e.g., U.S. Pat. No. 5,478,925, which is herein incorporated by reference in its entirety). Additionally, other techniques known in the art may be applied to generate liposomes containing the peptides components desired to be contained in the multimer of the invention (see, e.g., U.S. Pat. No. 5,478,925, which is herein incorporated by reference in its entirety).

The peptides expressly provided herein, as well as the fragments thereof, may further comprise linker elements that facilitate the attachment of the fragments to other molecules, amino acids, or polypeptide sequences. The linkers can also be used to attach the peptides, or fragments thereof, to solid support matrices for use in affinity purification protocols. Non-limiting examples of “linkers” suitable for the practice of the invention include chemical linkers (such as those sold by Pierce, Rockford, Ill.), or peptides that allow for the connection combinations of peptides (see, for example, linkers such as those disclosed in U.S. Pat. Nos. 6,121,424, 5,843,464, 5,750,352, and 5,990,275, hereby incorporated by reference in their entirety).

In other embodiments, the linker element can be an amino acid sequence (a peptide linker). In some embodiments, the peptide linker has one or more of the following characteristics: a) it allows for the free rotation of the peptides that it links (relative to each other); b) it is resistant or susceptible to digestion (cleavage) by proteases; and c) it does not interact with the peptides it joins together. In various embodiments, a multimeric construct according to the subject invention includes a peptide linker and the peptide linker is 5 to 60 amino acids in length. More preferably, the peptide linker is 10 to 30, amino acids in length; even more preferably, the peptide linker is 10 to 20 amino acids in length. In some embodiments, the peptide linker is 17 amino acids in length.

Multimeric constructs of the subject invention can also comprise a series of repeating elements, optionally interspersed with other elements. As would be appreciated by one skilled in the art, the order in which the repeating elements occur in the multimeric polypeptide is not critical and any arrangement of the repeating elements as set forth herein can be provided by the subject invention. Thus, a “multimeric construct” according to the subject invention can provide a multimeric peptide comprising a series of peptides, or peptide fragments, that are, optionally, joined together by linker elements (either chemical linker elements or amino acid linker elements).

A “variant” or “variant peptide” (or peptide variant) is to be understood to designate peptides exhibiting, in relation to the peptides disclosed herein, certain modifications. These modifications can include a deletion, addition, or substitution of at least one amino acid (e.g., one, two, three or more amino acids), a truncation, an extension, a chimeric fusion (fusion protein), a mutation, or polypeptides exhibiting post-translational modifications. These modifications can occur anywhere in the peptide, e.g., one or both ends and/or in the middle. Among these homologous variant peptides, are those comprising amino acid sequences exhibiting between at least (or at least about) 20.00% to 99.99% (inclusive) identity to the full length, native, or naturally occurring polypeptide are another aspect of the invention. The aforementioned range of percent identity is to be taken as including, and providing written description and support for, any fractional percentage, in intervals of 0.01%, between 20.00% and, up to, including 99.99%. These percentages are purely statistical and differences between two polypeptide sequences can be distributed randomly and over the entire sequence length. Thus, variant peptides can have 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent identity with the peptide sequences of the instant invention. In a preferred embodiment, a variant or modified peptide exhibits at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent identity to the reference peptide. The percent identity is calculated with reference to the full-length polypeptide or the length of the fragment of a particular SEQ ID NO: that is identified.

Preferably, the variant peptides retain at least one of the biological activities associated with the reference peptide (for example, the ability to: 1) specifically bind to antibodies specific for the full-length peptide from which the fragment was derived; 2) interfere with protein-protein interaction; 3) cause apoptosis in target cells (e.g., malignant cells), regardless of mechanism of action).

For example, one or more amino acid residues within the sequence can be substituted by another amino acid of a similar polarity which acts as a functional equivalent, resulting in a silent alteration. In one aspect of the present invention, conservative substitutions for an amino acid within the sequence may be selected from other members of the class to which the amino acid belongs (see Table 5). Conservative substitutions also include substitutions by amino acids having chemically modified side chains that do not eliminate the biological function of the resulting variant.

TABLE 5

| Class of Amino Acid | Examples of Amino Acids |
|---|---|
| Nonpolar | Ala, Val, Leu, Ile, Pro, Met, Phe, Trp |
| Uncharged Polar | Gly, Ser, Thr, Cys, Tyr, Asn, Gln |
| Acidic | Asp, Glu |
| Basic | Lys, Arg, His |

For example, one or more amino acids in the PPIIs of formulas I-X and Tables 1 and 2 can be conservatively substituted with an amino acid in which the cyclic peptide retains at least one of the biological activities associated with the reference peptide (for example, the ability to: 1) specifically bind to antibodies specific for the full-length peptide from which the fragment was derived; 2) interfere with protein-protein interaction; 3) cause apoptosis in target cells (e.g., malignant cells), regardless of mechanism of action).

Fusion proteins according to the subject invention comprise one or more heterologous peptide sequences (e.g., tags that facilitate purification of the peptides of the invention (see, for example, U.S. Pat. No. 6,342,362, hereby incorporated by reference in its entirety; Altendorf et al. [1999-WWW, 2000]“Structure and Function of the Fₒ Complex of the ATP Synthase from Escherichia Coli,” J. of Experimental Biology 203:19-28, The Co. of Biologists, Ltd., G. B.; Baneyx [1999]“Recombinant Protein Expression in Escherichia coli,” Biotechnology 10:411-21, Elsevier Science Ltd.; Eihauer et al. [2001]“The FLAG™ Peptide, a Versatile Fusion Tag for the Purification of Recombinant Proteins,” J. Biochem Biophys Methods 49:455-65; Jones et al. [1995] J. Chromatography 707:3-22; Jones et al. [1995]“Current Trends in Molecular Recognition and Bioseparation,” J. of Chromatography A. 707:3-22, Elsevier Science B. V.; Margolin [2000]“Green Fluorescent Protein as a Reporter for Macromolecular Localization in Bacterial Cells,” Methods 20:62-72, Academic Press; Puig et al. [2001]“The Tandem Affinity Purification (TAP) Method: A General Procedure of Protein Complex Purification,” Methods 24:218-29, Academic Press; Sassenfeld [1990] “Engineering Proteins for Purification,” TibTech 8:88-93; Sheibani [1999] “Prokaryotic Gene Fusion Expression Systems and Their Use in Structural and Functional Studies of Proteins,” Prep. Biochem. & Biotechnol. 29 (1):77-90, Marcel Dekker, Inc.; Skerra et al. [1999] “Applications of a Peptide Ligand for Streptavidin: the Strep-tag”, Biomolecular Engineering 16:79-86, Elsevier Science, B. V.; Smith [1998]“Cookbook for Eukaryotic Protein Expression Yeast, Insect, and Plant Expression Systems,” The Scientist 12 (22):20; Smyth et al. [2000] “Eukaryotic Expression and Purification of Recombinant Extracellular Matrix Proteins Carrying the Strep II Tag”, Methods in Molecular Biology, 139:49-57; Unger [1997]“Show Me the Money: Prokaryotic Expression Vectors and Purification Systems,” The Scientist 11 (17):20, each of which is hereby incorporated by reference in their entireties), or commercially available tags from vendors such as such as STRATAGENE (La Jolla, Calif.), NOVAGEN (Madison, Wis.), QIAGEN, Inc., (Valencia, Calif.), or InVitrogen (San Diego, Calif.).

In other embodiments, peptides of the subject invention can be fused to heterologous polypeptide sequences that have adjuvant activity (a polypeptide adjuvant). Non-limiting examples of such polypeptides include heat shock proteins (hsp) (see, for example, U.S. Pat. No. 6,524,825, the disclosure of which is hereby incorporated by reference in its entirety).

Peptides as described herein may be synthesized by methods well known in the art, including recombinant DNA methods and chemical synthesis. Chemical synthesis may generally be performed using standard solution phase or solid phase peptide synthesis techniques, in which a peptide linkage occurs through the direct condensation of the amino group of one amino acid with the carboxy group of the other amino acid with the elimination of a water molecule. Peptide bond synthesis by direct condensation, as formulated above, requires suppression of the reactive character of the amino group of the first and of the carboxyl group of the second amino acid. The masking substituents must permit their ready removal, without inducing breakdown of the labile peptide molecule.

In solution phase synthesis, a wide variety of coupling methods and protecting groups may be used (see Gross and Meienhofer, eds., “The Peptides: Analysis, Synthesis, Biology,” Vol. 1-4 (Academic Press, 1979); Bodansky and Bodansky, “The Practice of Peptide Synthesis,” 2d ed. (Springer Verlag, 1994)). In addition, intermediate purification and linear scale up are possible. Those of ordinary skill in the art will appreciate that solution synthesis requires consideration of main chain and side chain protecting groups and activation method. In addition, careful segment selection is necessary to minimize racemization during segment condensation. Solubility considerations are also a factor.

Solid phase peptide synthesis uses an insoluble polymer for support during organic synthesis. The polymer-supported peptide chain permits the use of simple washing and filtration steps instead of laborious purifications at intermediate steps. Solid-phase peptide synthesis may generally be performed according to the method of Merrifield et al., J. Am. Chem. Soc., 1963, 85:2149, which involves assembling a linear peptide chain on a resin support using protected amino acids. Solid phase peptide synthesis typically utilizes either the Boc or Fmoc strategy, which are well known in the art.

Those of ordinary skill in the art will recognize that, in solid phase synthesis, deprotection and coupling reactions must go to completion and the side-chain blocking groups must be stable throughout the synthesis. In addition, solid phase synthesis is generally most suitable when peptides are to be made on a small scale.

Acetylation of the N-terminal can be accomplished by reacting the final peptide with acetic anhydride before cleavage from the resin. C-amidation is accomplished using an appropriate resin such as methylbenzhydrylamine resin using the Boc technology.

The peptides disclosed herein may be modified by attachment of a second molecule that confers a desired property upon the peptide, such as increased half-life in the body, for example, pegylation. Such modifications also fall within the scope of the term “variant” as used herein.

Covalent attachment of a molecule or solid support may generally be achieved by first reacting the support material with a bifunctional reagent that will also react with a functional group, such as a hydroxyl, thiol, carboxyl, ketone or amino group, on the modulating agent. A preferred method of generating a linkage is via amino groups using glutaraldehyde. A peptide may be linked to cellulose via ester linkages. Similarly, amide linkages may be suitable for linkage to other molecules such as keyhole limpet hemocyanin or other support materials.

Although PPIIs as described herein may preferentially bind to specific tissues or cells, and thus may be sufficient to target a desired site in vivo, it may be beneficial for certain applications to include an additional targeting agent. Accordingly, a targeting agent may also, or alternatively, be linked to a PPII to facilitate targeting to one or more specific tissues. As used herein, a “targeting agent,” may be any substance (such as a compound or cell) that, when linked to a PPII, enhances the transport of the inhibitor to a target tissue, thereby increasing the local concentration of the PPII. Targeting agents include antibodies or fragments thereof, receptors, ligands and other molecules that bind to cells of, or in the vicinity of, the target tissue. Known targeting agents include serum hormones, antibodies against cell surface antigens, lectins, adhesion molecules, tumor cell surface binding ligands, steroids, cholesterol, lymphokines, fibrinolytic enzymes and those drugs and proteins that bind to a desired target site.

For certain embodiments, it may be beneficial to also, or alternatively, link a drug to a PPII. As used herein, the term “drug” refers to any bioactive agent intended for administration to a human or non-human mammal to prevent or treat a disease or other undesirable condition. Drugs include hormones, growth factors, proteins, peptides and other compounds.

As used in this specification, the singular forms “a”, “an”, and “the” include plural reference unless the context clearly dictates otherwise. Thus, for example, a reference to “a compound” includes more than one such compound. Reference to “PPII” includes more than one such PPII. A reference to “a peptoid body” includes more than one such peptoid body, and so forth.

Samples may be arrayed on a substrate, or multiple substrates can be utilized, for multiplex detection or analysis. “Arraying” refers to the act of organizing or arranging members of a library (e.g., an array of different samples), or other collection, into a logical or physical array. Thus, an “array” refers to a physical or logical arrangement of, e.g., biological samples. A physical array can be any “spatial format” or physically gridded format” in which physical manifestations of corresponding library members are arranged in an ordered manner, lending itself to combinatorial screening. For example, samples corresponding to individual or pooled members of a sample library can be arranged in a series of numbered rows and columns, e.g., on a multi-well plate. Similarly, binding moieties can be plated or otherwise deposited in microtitered, e.g., 96-well, 384-well, or -1536 well, plates (or trays). In some embodiments, peptoid bodies of the invention are attached to the substrate.

As used herein, the terms solid “substrate”, “support”, and “surface” refer to a solid phase which is a porous or non-porous water insoluble material that can have any of a number of shapes, such as strip, rod, particle, beads, or multi-welled plate. In some embodiments, the support has a fixed organizational support matrix that preferably functions as an organization matrix, such as a microtiter tray. Solid support materials include, but are not limited to, cellulose, polysaccharide such as Sephadex, glass, polyacryloylmorpholide, silica, controlled pore glass (CPG), polystyrene, polystyrene/latex, polyethylene such as ultra high molecular weight polyethylene (UPE), polyamide, polyvinylidine fluoride (PVDF), polytetrafluoroethylene (PTFE; TEFLON), carboxyl modified teflon, nylon, nitrocellulose, and metals and alloys such as gold, platinum and palladium. The solid support can be biological, non-biological, organic, inorganic, or a combination of any of these, existing as particles, strands, precipitates, gels, sheets, pads, cards, strips, dipsticks, test strips, tubing, spheres, containers, capillaries, pads, slices, films, plates, slides, etc., depending upon the particular application. Preferably, the solid support is planar in shape, to facilitate contact with a biological sample such as urine, whole blood, plasma, serum, peritoneal fluid, or ascites fluid. Other suitable solid support materials will be readily apparent to those of skill in the art. The solid support can be a membrane, with or without a backing (e.g., polystyrene or polyester card backing), such as those available from Millipore Corp. (Bedford, Mass.), e.g., HI-FLOW Plus membrane cards. The surface of the solid support may contain reactive groups, such as carboxyl, amino, hydroxyl, thiol, or the like for the attachment of nucleic acids, proteins, etc. Surfaces on the solid support will sometimes, though not always, be composed of the same material as the support. Thus, the surface can be composed of any of a wide variety of materials, such as polymers, plastics, resins, polysaccharides, silica or silica-based materials, carbon, metals, inorganic glasses, membranes, or any of the aforementioned support materials (e.g., as a layer or coating). In some embodiments, the substrate comprises a resin.

As used herein, the terms “label” and “tag” refer to substances that may confer a detectable signal, and include, but are not limited to, enzymes such as alkaline phosphatase, glucose-6-phosphate dehydrogenase, and horseradish peroxidase, ribozyme, a substrate for a replicase such as QB replicase, promoters, dyes, quantum dots, fluorescers, such as fluorescein, isothiocynate, rhodamine compounds, phycoerythrin, phycocyanin, allophycocyanin, o-phthaldehyde, and fluorescamine, chemiluminescers such as isoluminol, sensitizers, coenzymes, enzyme substrates, radiolabels, particles such as latex or carbon particles, liposomes, cells, etc., which may be further labeled with a dye, catalyst or other detectable group. The PPII or potential target protein or cell may be labeled. In some embodiments, the label is a quantum dot. For example, binding of a peptoid body with a quantum dot-labeled target cell or protein may result in detectable fluorescence.

As used herein, in the context of peptoid-peptide hybrids, the term “target” refers to a target molecule, such as a target protein or other biological molecule. In some embodiments, the target is an extracellular molecule such as a molecule on the outer surface of a cell, such as a membrane protein (integral or peripheral membrane protein). The peptoid body approach of the invention has the potential to change disease diagnoses and treatment, as an array of peptoid bodies can be used to repeatedly screen a wide range of targets followed by identification of easily produced targeting ligands economically. Table 1, above, provides a non-exclusive list of possible targeted and untargeted cells.

As used herein, the term “protein” refers to a sequence of amino acids of any length (two or more). Thus, the term is used interchangeably with the terms polypeptide and peptide.

### Exemplified Embodiments

### Embodiment 1

A peptoid-body, comprising a cyclic peptoid-peptide hybrid having a beta-hairpin-like conformation, comprising a plurality of alternating peptoid-peptide sequences, each having at least one peptoid residue and an amino acid residue, wherein the peptoid-peptide sequences form at least two antiparallel beta-strands between a plurality of linkers, and wherein at least one linker is a beta-turn promoter.

### Embodiment 2

The peptoid-body according to embodiment 1, wherein at least one of the linker is the amino acid residue from the condensation of the linker precursor of the structure:

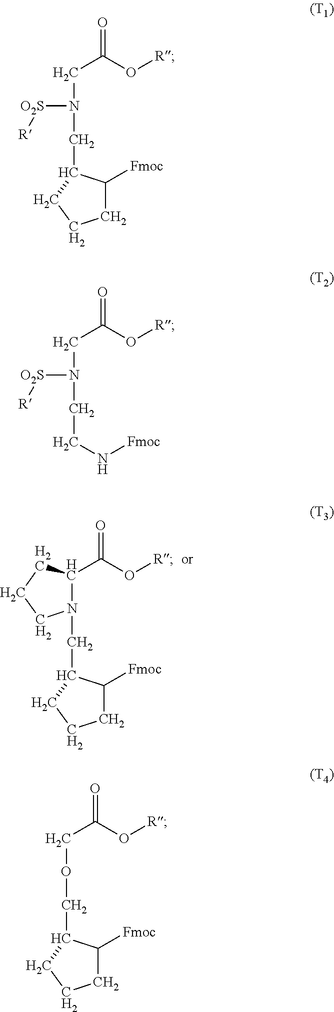

wherein where R′ is an organic group, or an organic bridging group attached to a resin or other substrate and R″ is H or a carboxylic acid protecting group.

### Embodiment 3

The peptoid-body according to embodiment 2, wherein R″ is t-butyl, allyl, or benzyl.

### Embodiment 4

The peptoid-body according to embodiment 2, wherein the organic bridging group attached to a resin or other substrate comprises a —NH(CH₂)₂-bridging group.

### Embodiment 5

The peptoid-body according to embodiment 1, wherein one of the linker is two peptoid residues.

### Embodiment 6

The peptoid-body according to embodiment 1, wherein the cyclic peptoid-peptide hybrid is:

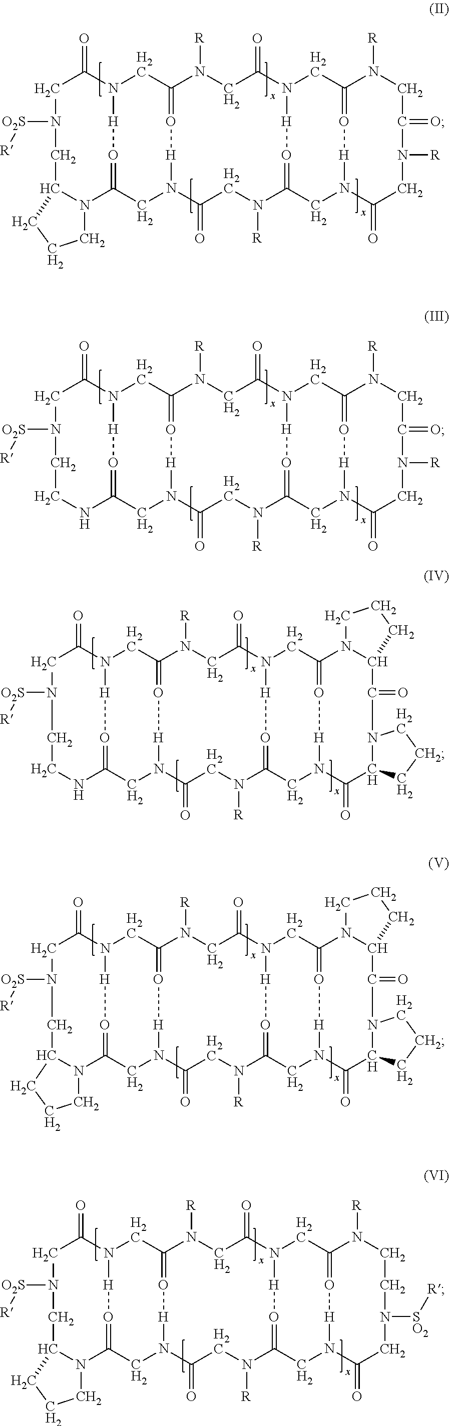

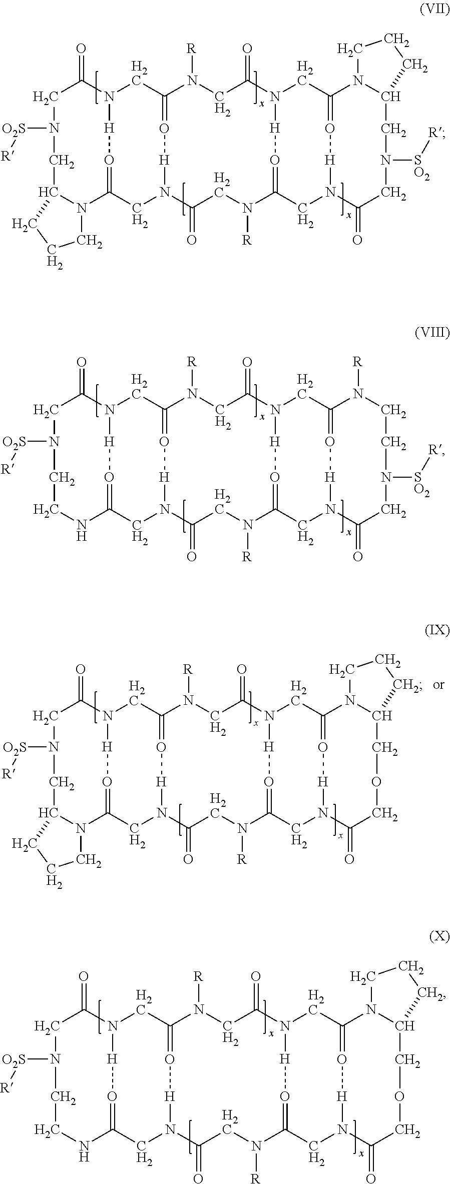

wherein R groups are independently organic groups, R′ is independently an organic group or an organic bridging group attached to a resin, and x is 1 to 3.

### Embodiment 7

The peptoid-body according to embodiment 6, wherein R is independently C₁-C₁₂ alkyl, C₁-C₁₂ hydroxyalkyl, C₁-C₁₂ aminoalkyl, C₁-C₁₂ carboxylic acid alkyl, C₂-C₁₂ alkyloxyalkyl, C₂-C₁₂ alkenyl, C₂-C₁₂ hydroxyalkenyl, C₂-C₁₂ aminoalkenyl, C₁-C₁₂ carboxylic acid alkenyl, C₃-C₁₄ alkyloxyalkenyl, C₆-C₁₄ aryl, C₆-C₁₄ hydroxyaryl, C₆-C₁₄ aminoaryl, C₆-C₁₄ carboxylic acid aryl, C₇-C₁₅ alkyloxyaryl, C₄-C₁₄ heteroaryl, C₄-C₁₄ hydroxyheteroaryl, C₄-C₁₄ aminoheteroaryl, C₄-C₁₄ carboxylic acid heteroaryl, C₅-C₁₅ alkoxyheteroaryl, C₇-C₁₅ alkylaryl, C₇-C₁₅ hydroxyalkylaryl, C₇-C₁₅ aminoalkylaryl, C₇-C₁₅ carboxylic acid alkylaryl, C₈-C₁₅ alkoxyalkylaryl, or any chemically transformed structure therefrom.

### Embodiment 8

The peptoid-body according to embodiment 7, wherein the chemically transformed structure comprises an ester, thioester, thiol, amide, or sulfonamide.

### Embodiment 9

The peptoid-body according to embodiment 6, wherein R is independently a residue of a primary amine: 4-aminopiperidine; ethanolamine; allylamine; 1;4-diaminobutane; piperponylamine; 4; (2-aminoethyl)benzene; isobutylamine; tryptamine; 4-morpholinoaniline; 5-amino-2-methoxypyridine; (R)-methylbenzylamine; 1-(2-aminopropyl)-2-pyrrolidinone; furfurylamine; benzylamine; 4-chlorobenzylamine; 4-methoxybenzylamine; methoxyethylamine. 2-aminoadipic acid; N-ethylasparagine; 3-aminoadipic acid; hydroxylysine; beta-alanine; allo-hydroxylysine propionic acid; 2-aminobutyric acid; 3-hydroxyproline; 4-Aminobutyric acid; 4-hydroxyproline piperidinic acid; 6-Aminocaproic acid; Isodesmosine; 2-Aminoheptanoic acid; allo-isoleucine; 2-aminoisobutyric acid; N-methylglycine; 3-aminoisobutyric acid; N-methylisoleucine; 2-Aminopimelic acid; 6-N-methyllysine; 2,4-diaminobutyric acid; N-methylvaline; desmosine; norvaline; 2,2′-diaminopimelic acid; norleucine; 2,3-diaminopropionic acid; ornithine; N-ethylglycine; or any protected equivalents thereof.

### Embodiment 10

The peptoid-body according to embodiment 6, wherein at least one R is a residue of 4-aminopiperidine.

### Embodiment 11

The peptoid-body according to embodiment 1, wherein all of the amino acid residues are glycine residues.

### Embodiment 12

An array of peptoid-bodies, comprising a multiplicity of peptoid-bodies according to embodiment 1, each comprising an organic bridging group attached to a resin or other substrate, wherein each of the peptide bodies are positioned in a spatially addressable array on a support (e.g., an array of wells on a plate), wherein each of the peptoid-bodies differs in structure, wherein upon exposure to differentially expressed extracellular proteins associated with a diseased cell are potentially identified by preferential binding to at least one of the multiplicity of peptoid-bodies.

### Embodiment 13

The array of peptoid-bodies according to embodiment 12, wherein each well comprises peptoid-bodies that differ only by the organic bridging group, wherein at least one bridging group is cleavable to release its cyclic peptoid-peptide hybrid upon cleavage and at least one bridging group is stable to retain its cyclic peptoid-peptide hybrid.

### Embodiment 14

The array of peptoid-bodies according to embodiment 12, wherein the spatially addressable array comprises 345 wells.

### Embodiment 15

The array of peptoid-bodies according to embodiment 12, wherein the support is a PP reservoir covered with a PEEK film, and wherein the resin or other substrate comprises a bead that chemically bonds with PEEK.

### Embodiment 16

A method of preparing a peptoid-body according to embodiment 1, comprising:
a) deprotecting an amine of a substrate bound linker by the removal of an Fmoc group to form a terminal amine;
b) forming an amide by reaction of the terminal amine with bromoacetic anhydride;
c) promoting nucleophilic substitution with 2,4-dimethoxybenzylamine (H₂NDMB) to generate a mono-N-protected glycine unit having a subsequent terminal amine;
d) repeating step b) wherein the terminal amine is the subsequent terminal amine;
e) promoting nucleophilic substitution with a primary amine to generate a subsequent terminal amine;
f) repeating step d) and step c);
g) optionally repeating each of steps d) through f) one or two times wherein the primary amine is the same or different from other primary amine(s);
h) formation of an amide formation of the by reaction of the subsequent terminal amine and a carboxylic end of a linker of the structure:

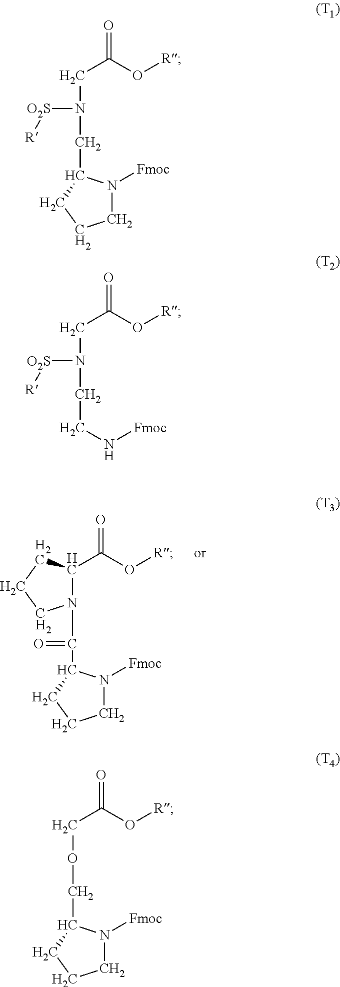

wherein where R′ is an organic group and R″ is H or a carboxylic acid protecting group and, subsequently deprotecting an amine of the linker by the removal of an Fmoc group to form a subsequent terminal amine, or repeating steps d) and e) twice;
i) repeating step d);
j) repeating steps c) through g);
k) reacting the subsequent terminal amine and a carboxylic end of the substrate bound linker to form a cyclic; and
l) removing the 2,4-dimethoxybenzyl (DMB) groups and any other protecting groups to yield a substrate bound peptoid body.

### Embodiment 17

The method according to embodiment 16, wherein the primary amine is RNH₂ wherein R is C₁-C₁₂ alkyl, C₁-C₁₂ hydroxyalkyl, C₁-C₁₂ aminoalkyl, C₁-C₁₂ carboxylic acid alkyl, C₂-C₁₂ alkyloxyalkyl, C₂-C₁₂ alkenyl, C₂-C₁₂ hydroxyalkenyl, C₂-C₁₂ aminoalkenyl, C₁-C₁₂ carboxylic acid alkenyl, C₃-C₁₄ alkyloxyalkenyl, C₆-C₁₄ aryl, C₆-C₁₄ hydroxyaryl, C₆-C₁₄ aminoaryl, C₆-C₁₄ carboxylic acid aryl, C₇-C₁₅ alkyloxyaryl, C₄-C₁₄ heteroaryl, C₄-C₁₄ hydroxyheteroaryl, C₄-C₁₄ aminoheteroaryl, C₄-C₁₄ carboxylic acid heteroaryl, C₅-C₁₅ alkoxyheteroaryl, C₇-C₁₅ alkylaryl, C₇-C₁₅ hydroxyalkylaryl, C₇-C₁₅ aminoalkylaryl, C₇-C₁₅ carboxylic acid alkylaryl, C₈-C₁₅ alkoxyalkylaryl, or any chemically transformed structure therefrom.

### Embodiment 18

The method according to embodiment 16, wherein the primary amine is 4-aminopiperidine; ethanolamine; allylamine; 1;4-diaminobutane; piperponylamine; 4; (2-aminoethyl)benzene; isobutylamine; tryptamine; 4-morpholinoaniline; 5-amino-2-methoxypyridine; (R)-methylbenzylamine; 1-(2-aminopropyl)-2-pyrrolidinone; furfurylamine; benzylamine; 4-chlorobenzylamine; 4-methoxybenzylamine; methoxyethylamine. 2-aminoadipic acid; N-ethylasparagine; 3-aminoadipic acid; hydroxylysine; beta-alanine; allo-hydroxylysine propionic acid; 2-aminobutyric acid; 3-hydroxyproline; 4-Aminobutyric acid; 4-hydroxyproline piperidinic acid; 6-Aminocaproic acid; Isodesmosine; 2-Aminoheptanoic acid; allo-isoleucine; 2-aminoisobutyric acid; N-methylglycine; 3-aminoisobutyric acid; N-methylisoleucine; 2-Aminopimelic acid; 6-N-methyllysine; 2,4-diaminobutyric acid; N-methylvaline; desmosine; norvaline; 2,2′-diaminopimelic acid; norleucine; 2,3-diaminopropionic acid; ornithine; N-ethylglycine; or any protected equivalents thereof.

### Embodiment 19

A method of identifying an inhibiting peptoid-body according to embodiment 1, comprising contacting an array of peptoid-bodies according to embodiment 12 with a sample comprising a target; and identifying where binding to one or more peptoid-bodies to the target is indicated.

### Embodiment 20

The method of embodiment 19, wherein the contacting is carried out on a substrate, wherein the target comprises cells and/or proteins, and wherein the identifying comprises identifying a well on the substrate where binding to target in the sample is indicated.

### Embodiment 21

The method of embodiment 19 or 20, wherein the identifying comprises detecting a detectable label attached to the target.

### Embodiment 22

The method of embodiment 19 or 20, wherein the identifying comprises using fluorescence from at least one quantum dot-labeled cell and/or quantum dot-labeled protein.

### Embodiment 23

A pharmaceutical composition, comprising a peptoid-body according to any one of embodiments 1-11.

### Embodiment 24

The pharmaceutical according to embodiment 23, wherein the peptoid-body is identified by the method of embodiment 19.

### Embodiment 25

The pharmaceutical composition according to embodiment 23, further comprising a pharmaceutically acceptable carrier.

### Embodiment 26

The pharmaceutical composition according to embodiment 23, further comprising one or more other anti-cancer agents.

### Embodiment 27

A method for treating a disorder in a subject, comprising administering an effective amount of a pharmaceutical composition according to embodiment 23 to the subject.

### Embodiment 28

The method according to embodiment 27, wherein the disorder is an oncological disorder.

### Embodiment 29

The method according to embodiment 27, wherein the disorder is an infection.

### Embodiment 30

The method according to embodiment 29, wherein the infection is malaria.

### Embodiment 31

The method according to embodiment 27, wherein the disorder is an immunoregulatory abnormality.

### Embodiment 32

The method according to embodiment 31, wherein the immunoregulatory abnormality is an autoimmune or chronic inflammatory disease.

### Embodiment 33

The method according to embodiment 28, wherein the method further comprises administering an effective amount of one or more other anti-cancer agents to the subject before, during, or after administration of the cancer inhibitor.

### Embodiment 34

A method for inducing apoptosis or inhibiting the growth of a cell, comprising contacting the cell with an effective amount of a peptoid body in vitro or in vivo according to any one of embodiments 1-11.

### Embodiment 35

The method according to embodiment 34, wherein the cell is a cancer cell.

### Embodiment 36

The method according to embodiment 34, wherein the cell is a pathogen-infected cell.

### Embodiment 37

The method according to embodiment 36, wherein the cell is a malaria-infected cell.

### Embodiment 38

The method according to embodiment 34, wherein the method further comprises contacting the cell with an effective amount of one or more other anti-cancer agents before, during, or after contacting with the cancer inhibitor.

The practice of the present invention can employ, unless otherwise indicated, conventional techniques of molecular biology, microbiology, recombinant DNA technology, electrophysiology, and pharmacology that are within the skill of the art. Such techniques are explained fully in the literature (see, e.g., Sambrook, Fritsch & Maniatis, Molecular Cloning: A Laboratory Manual, Second Edition (1989); DNA Cloning, Vols. I and II (D. N. Glover Ed. 1985); Perbal, B., A Practical Guide to Molecular Cloning (1984); the series, Methods In Enzymology (S. Colowick and N. Kaplan Eds., Academic Press, Inc.); Transcription and Translation (Hames et al. Eds. 1984); Gene Transfer Vectors For Mammalian Cells (J. H. Miller et al. Eds. (1987) Cold Spring Harbor Laboratory, Cold Spring Harbor, N.Y.); Scopes, Protein Purification: Principles and Practice (2nd ed., Springer-Verlag); and PCR: A Practical Approach (McPherson et al. Eds. (1991) IRL Press)), each of which are incorporated herein by reference in their entirety.

Experimental controls are considered fundamental in experiments designed in accordance with the scientific method. It is routine in the art to use experimental controls in scientific experiments to prevent factors other than those being studied from affecting the outcome.

All patents, patent applications, provisional applications, and publications referred to or cited herein, supra or infra, are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Example 1

### Cyclic Peptoid-Peptide Hybrids

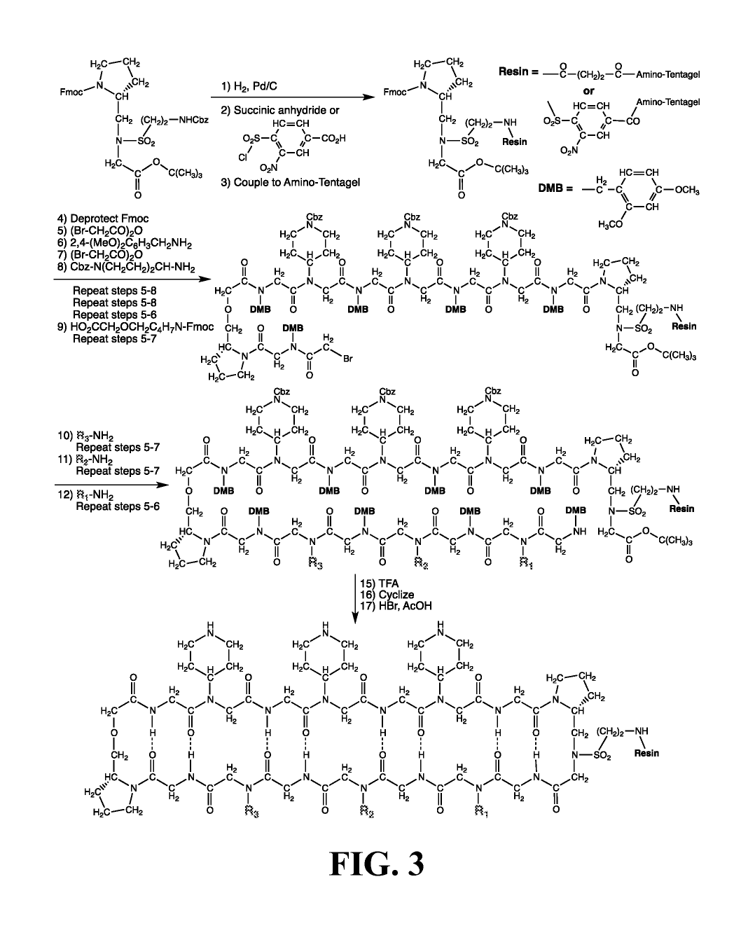

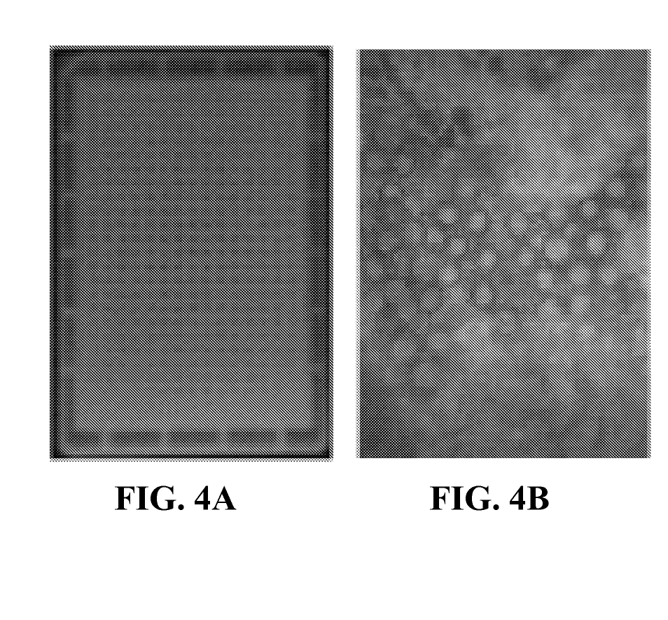

A cyclic b-hairpin-like scaffold with 3 variable peptoid side chains, 3 invariant water-solubilizing peptoid side chains, 8 glycines, and 2 b-turn promoters is prepared, as shown in FIG. 3 using a standard PP flat-bottom reservoir with a 300-micron film of polyether ether ketone [PEEK] cut to fit the reservoir bottom and a precisely machined 0.5-inch thick rectangular wafer of PP with 345 holes [3-mm diameter each] assembled, as shown in FIG. 4A). PEEK is solvent resistant and is surface-modified by reaction with chlorosulfonic acid to give surface arylsulfonyl chlorides. The treated surface reacts with mino-Tentagel beads to yield stable arylsulfonylamide linkages, as shown in 5b) as a 5× magnified photomicrograph of amino-Tentagel beads on the surface-modified PEEK film. Unreacted amino-groups of the Tentagel beads undergo sequential attachment of the invariant portions of the library on the arrayed beads, as shown in FIG. 4. For 115 different primary amines, 38.33 slides have an identical R₃-primary amine, 115 spots of those slides [⅓ of a slide] will have identical R₂-primary amine, and are individual reacted with 115 different R₁-primary amines will give 345 different b-hairpin-like scaffold peptoid bodies/well as an arrayed library in PP reservoirs ready for automated multi-plate washing and screening where the screening hit location defines the hit sequence.

The scaffold varies 3 positions along one outside edge of the b-hairpin-like structure at the peptoid positions and alternating glycines give a b-sheet-like hydrogen-bonding network. With 115 different variable groups, 115×115×115 or over 1.5 million different b-hairpin-like scaffolds are made. These b-hairpin-like scaffolds are synthesized in place, using a geometrical format that allows automated pipetting of reagents like the commercialized 384 well-plates except in a novel PEEK-coated PP reservoir with 345-wells. The PP arrays are screens to find specific beta-hairpin-like scaffolds that bind a carbonic anhydrase IX (CAIX)-overexpressing hypoxic cell (red fluorescently-labeled) and counter-screened with the same cell under normoxic conditions lacking CAIX overexpression (green fluorescently-labeled). The easier hit-to-lead optimization for the beta-hairpin-like scaffold hits allows CAIX diagnostic imaging and/or identification of therapeutic targeting agents. More than 29,000 commercially available primary amines can be used to optimize the side R groups for each variable peptoid position. Using cleavable and stable linkers, as shown in Step 2 of FIG. 3, synthetic fidelity is determined by cleavage and HPLC-MS analysis of released library members, where remaining stable library members arrayed in that same spot remain to be screened without requiring synthetic fidelity testing.

Reservoirs are constructed from a 300-micron thick PEEK film [APTIV Film, 1000 Series, from Victrex USA, Inc., West Conshohocken, Pa.] with a low profile flat bottom PP reservoirs [BioTech Solutions Disposable PP Reservoirs; Laboratory Supply Distributors, Millville, N.J.]. Precisely machined PP wafers that fit tightly, but easily within the PP reservoirs with 345 arrayed holes each 3-mm in diameter. The surface-modified PEEK permits capture of amino-Tentagel beads, as shown on FIG. 5b).

HPLC-MS analysis verified that the chemical steps shown in FIG. 4 resulted in the desired sequence. Hits are characterized according to the chemotype of the variable peptoid side chains in each of the different positions. One set of possibilities are: 1) hydrophobic, aliphatic; 2) hydrophobic, electron rich aromatic; 3) hydrophobic, electron deficient aromatic; 4) amphipathic, aliphatic; 5) amphipathic, aromatic; 6) polar, uncharged; 7) polar, negatively charged, 8) polar, positively charged; and 9) polar, zwitterionic. The libraries are focused by varying a single variable peptoid side chain at a time using progressively finer changes in the varied single side chain, to optimize binding. Where a particular chemotype is preferred by random screening hits 1) and 2) from the list above, the libraries are focused by fine tuning those position using structurally different but similar chemotypes. When that side chain is optimized as determined by competitive binding or binding affinity studies, each of the remaining variable peptoid positions are optimize in turn.

Since all of the hits [up to 345 arrayed sites] on the new plate have some binding affinity for the targeted cells competively hits with the greatest relative binding are selected for detailed binding affinity analysis. Counter-screening using cells that do not express the target can be done simultaneously or sequentially to further select leads with both the desired binding affinity and binding specificity. Synthesis of a single plate filled or partially filled with hits for the targeted cells and the fast arrayed screening allows multiple rounds of assays in a shorter period than is possible with on bead one color libraries that are not arrayed. When less than 345 arrayed sites are needed, arrayed spots are positioned as far apart as possible to reduce light bleed-over.

The detailed synthesis of the 115×115×115 library is outlined in FIG. 4. Step 1 is the selective deprotection of the taurine-derived beta-turn. Step 2 is attachment to 20 mole % of the 3-nitro-4-sulfonylamidebenzoic acid linker and 80 mole % of the succinic acid linker to give each arrayed spot a cleavable and a stable amino-Tentagel linkage. The 3-nitro-4-sulfonylamidebenzoic acid linker can undergo thiolate promoted cleavage from the resin to verify synthetic fidelity by HPLC-MS analysis. In Step 3, the coupling to arrayed amino-Tentagel resin beads from Rappe Polymere allows synthesis using a variety of organic solvents and on bead assays in aqueous media. Steps 4-8 are standard peptoid synthesis steps except for the use of DMB as a protecting group of the secondary amide of the “glycine-like” monomers. In contrast to switching between Fmoc-glycine and peptoid synthesis steps where the Fmoc-glycine coupling is slow, the bromoacetic anhydride coupling is rapid and facile even though it includes an additional S_{N}2 synthesis step. Trace amounts of acylation of the “glycine-like” secondary amide by bromoacetic anhydride. Acylation of the secondary amine in Step 9 is very efficient. Steps 10-13 are standard for peptoid synthesis. R₃-, R₂-, and R₁-side chains can be any primary amine that are unreactive with primary amines and bromoacetic anhydride, and when protected, are preferably able to be deprotected by HBr/acetic acid for efficient concomitant deprotection. In Step 15, the terminal DMB and the other DMB groups are removed to allow the complementary hydrogen-bonding to facilitate the cyclization step in Step 16. In Step 17, the Cbz-protecting groups are removed. If the peptoid-peptide hybrid is to be cleaved from resin, thiolate mediated-cleavage on the ortho-nitroarylsulfonamide linker can be employed, and the remaining stable library members arrayed in that spot continue to be screened and without requiring synthetic fidelity testing.

A dual-color cell adhesion assay is used to screen OBTC libraries, where marker positive cells were stained green and negative cells were stained red, as shown in FIG. 6. Beads that bind only green cells are considered positive hits. Quantum dot cell labeling system disclosed in Udugamasooriya et al. On-Bead Two-Color (OBTC) Cell Screen for Direct Identification of Highly Selective Cell Surface Receptor Ligands. Current protocols in chemical biology 2012 4 35-48, with Qtraker cell labeling kits [Invitrogen], for high intensity fluorescence and increased signal-to-noise. MCF-7 cells grown in hypoxic conditions are caused to unadhere from the culture flask using Cellstripper reagent [Cellgro] and compound arrays are incubated with labeled MCF-7 cells inside the hypoxia chamber, gently washed to remove nonadherent cells and fluorescence read-out using the EnVision 2013 Multilabel Platereader [PerkinElmer] with the plate stacker housed in a High-Throughput Screening facility. The plate reader employs fluorescence filter sets that are optimal for Qtraker labeling kits.

Plates are subsequently stripped of cells and re-screened using labeled MCF-7 cells incubated in normoxic conditions. Spots on the array that fluoresce in the presence of the CAIX positive cells, but not in the negative cells, are considered hits. Screening and counter-screening can be carried out simultaneously.

Optimizations may be required for duration of cell-incubation on the array, stringency of washes, spacing of spots on the array due to bleed-over of fluorescent light from adjacent spots. Alternately, a grid with opaque sides can be placed over the array to limit light bleed-over.

A whole-cell assay is used for ligand discovery preferably over in vitro protein binding assays as the cell-surface topography represents the ultimate targeting landscape. For saturation binding, to determine the dissociation constant [K_{Da}], hit compounds are conjugated to a Eu-DTPA chelate and a dilution series of compound are added across a 96 well plate that has been uniformly seeded with MCF-7 breast cancer cells using a WellMate automated pipetting system [Matrix] and pre-incubated in hypoxic conditions to induce CAIX expression prior to screening. Following addition of test ligand and incubation, plates are washed using the ELx405 Select CW plate washer [BioTek] and DELFIA enhancement solution added [PerkinElmer] using the WellMate. Following incubation, time-resolved fluorescence [TRF] is read-out using the Victor X4 2030 Multilabel plate reader [PerkinElmer] and standard Eu TRF settings. This plate reader is equipped with a plate stacker. The HTS facility has a Biomek FXP robotic liquid handler with a span 8 for generating dilution series across plates for the binding assays.

Once a Eu-chelate conjugate is shown to have sub-micromolar binding affinity, the labeled compound is used to screen unlabeled libraries containing variations of the compound by competition binding assay to discover lead compounds with improved properties, i.e. increased binding affinity with a benchmark of obtaining a <100 nM inhibition constant [Ki], increased solubility and biostability. For the competition assay, a uniform concentration of the competing Eu-labeled compound is added across the plate and decreasing concentrations of each test ligand are added across the plate, followed by washes, incubation with enhancement solution and TRF readout. Finally, recombinant CAIX binding assays are necessary to specifically select CAIX binders from other potentially interesting whole cell binding assay hits that may target another overexpressed protein on the surface of the hypoxic MCF-7 cells. Five of the top hits are biotinylated and bound to commercially available streptavidin plates for the GE Healthcare BiaCore T100 SPR Kinetics System.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

We claim:

1. A peptoid-body, comprising a cyclic peptoid-peptide hybrid having a beta-hairpin-like conformation, comprising a plurality of alternating peptoid-peptide sequences, each having at least one peptoid residue and an amino acid residue, wherein the peptoid-peptide sequences form at least two antiparallel beta-strands between a plurality of linkers, and wherein at least one linker is a beta-turn promoter.

2. The peptoid-body according to claim 1, wherein at least one of the linker is the amino acid residue from the condensation of the linker precursor of the structure: 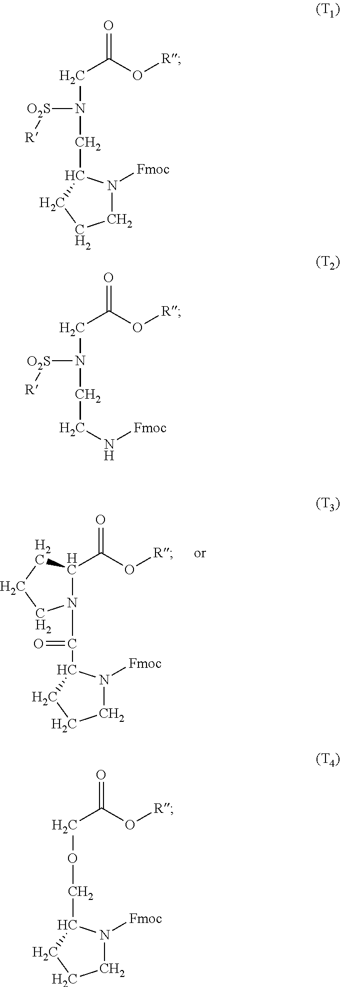
wherein where R′ is an organic group, or an organic bridging group attached to a resin or other substrate and R″ is H or a carboxylic acid protecting group.

3. The peptoid-body according to claim 2, wherein R″ is t-butyl, allyl, or benzyl.

4. The peptoid-body according to claim 2, wherein the organic bridging group attached to a resin or other substrate comprises a —NH(CH₂)₂— bridging group.

5. The peptoid-body according to claim 1, wherein one of the linkers is two peptoid residues.

6. The peptoid-body according to claim 1, wherein the cyclic peptoid-peptide hybrid is: 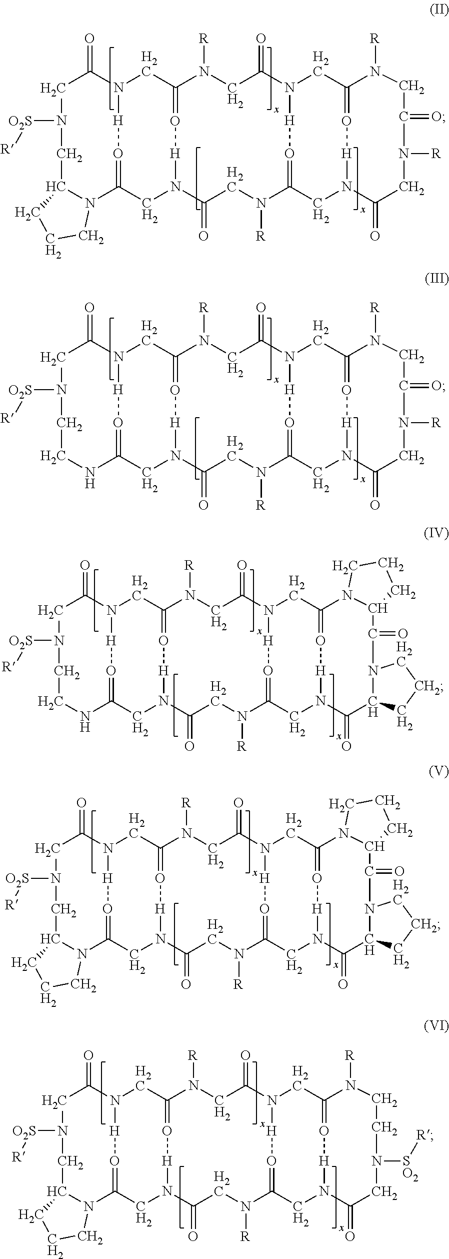 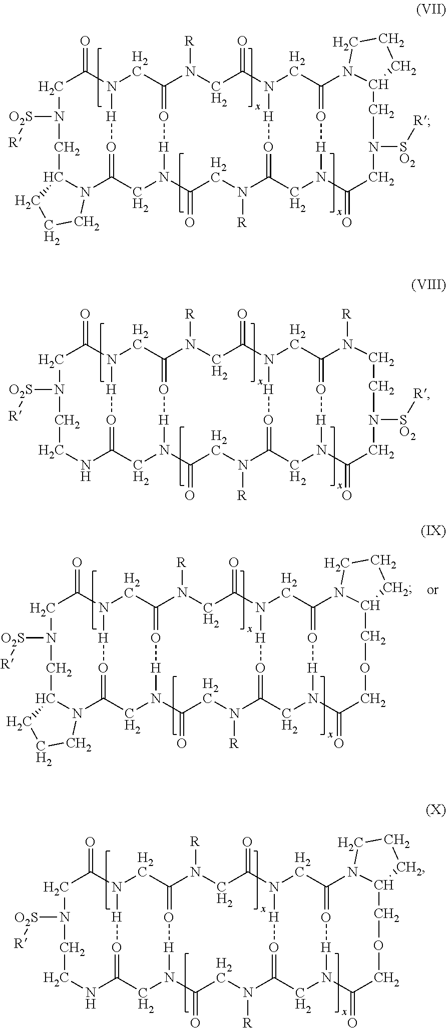
wherein R groups are independently organic groups, R′ is independently an organic group or an organic bridging group attached to a resin, and x is 1 to 3.

7. The peptoid-body according to claim 6, wherein R is independently C₁-C₁₂ alkyl, C₁-C₁₂ hydroxyalkyl, C₁-C₁₂ aminoalkyl, C₁-C₁₂ carboxylic acid alkyl, C₂-C₁₂ alkyloxyalkyl, C₂-C₁₂ alkenyl, C₂-C₁₂ hydroxyalkenyl, C₂-C₁₂ aminoalkenyl, C₁-C₁₂ carboxylic acid alkenyl, C₃-C₁₄ alkyloxyalkenyl, C₆-C₁₄ aryl, C₆-C₁₄ hydroxyaryl, C₆-C₁₄ aminoaryl, C₆-C₁₄ carboxylic acid aryl, C₇-C₁₅ alkyloxyaryl, C₄-C₁₄ heteroaryl, C₄-C₁₄ hydroxyheteroaryl, C₄-C₁₄ aminoheteroaryl, C₄-C₁₄ carboxylic acid heteroaryl, C₅-C₁₅ alkoxyheteroaryl, C₇-C₁₅ alkylaryl, C₇-C₁₅ hydroxyalkylaryl, C₇-C₁₅ aminoalkylaryl, C₇-C₁₅ carboxylic acid alkylaryl, C₈-C₁₅ alkoxyalkylaryl, or any chemically transformed structure therefrom.

8. The peptoid-body according to claim 7, wherein the chemically transformed structure comprises an ester, thioester, thiol, amide, or sulfonamide.

9. The peptoid-body according to claim 6, wherein R is independently a residue of a primary amine: 4-aminopiperidine; ethanolamine; allylamine; 1;4-diaminobutane; piperponylamine; 4; (2-aminoethyl)benzene; isobutylamine; tryptamine; 4-morpholinoaniline; 5-amino-2-methoxypyridine; (R)-methylbenzylamine; 1-(2-aminopropyl)-2-pyrrolidinone; furfurylamine; benzylamine; 4-chlorobenzylamine; 4-methoxybenzylamine; methoxyethylamine; 2-aminoadipic acid; N-ethylasparagine; 3-aminoadipic acid; hydroxylysine; beta-alanine; allo-hydroxylysine propionic acid; 2-aminobutyric acid; 3-hydroxyproline; 4-Aminobutyric acid; 4-hydroxyproline piperidinic acid; 6-Aminocaproic acid; Isodesmosine; 2-Aminoheptanoic acid; allo-isoleucine; 2-aminoisobutyric acid; N-methylglycine; 3-aminoisobutyric acid; N-methylisoleucine; 2-Aminopimelic acid; 6-N-methyllysine; 2,4-diaminobutyric acid; N-methylvaline; desmosine; norvaline; 2,2′-diaminopimelic acid; norleucine; 2,3-diaminopropionic acid; ornithine; N-ethylglycine; or any protected equivalents thereof.

10. The peptoid-body according to claim 6, wherein at least one R is a residue of 4-aminopiperidine.

11. The peptoid-body according to claim 1, wherein all of the amino acid residues are glycine residues.

12. An array of peptoid-bodies, comprising a multiplicity of peptoid-bodies according to claim 1, each comprising an organic bridging group attached to a resin or other substrate, wherein each of the peptide bodies are positioned in a spatially addressable array on a support, wherein each of said peptoid-bodies differs in structure, wherein upon exposure to differentially expressed extracellular proteins associated with a diseased cell are potentially identified by preferential binding to at least one of the multiplicity of peptoid-bodies.

13. The array of peptoid-bodies according to claim 12, wherein each well comprises peptoid-bodies that differ only by the organic bridging group, wherein at least one bridging group is cleavable to release its cyclic peptoid-peptide hybrid upon cleavage and at least one bridging group is stable to retain its cyclic peptoid-peptide hybrid.

14. The array of peptoid-bodies according to claim 12, wherein the support is a PP reservoir covered with a PEEK film, and wherein the resin or other substrate comprises a bead that chemically bonds with PEEK.

15. A method of preparing a peptoid-body according to claim 1, comprising:
a) deprotecting an amine of a substrate bound linker by the removal of an Fmoc group to form a terminal amine;
b) forming an amide by reaction of the terminal amine with bromoacetic anhydride;
c) promoting nucleophilic substitution with 2,4-dimethoxybenzylamine (H₂NDMB) to generate a mono-N-protected glycine unit having a subsequent terminal amine;
d) repeating step b) wherein the terminal amine is the subsequent terminal amine;
e) promoting nucleophilic substitution with a primary amine to generate a subsequent terminal amine;
f) repeating step d) and step c);
g) optionally repeating each of steps d) through f) one or two times wherein the primary amine is the same or different from other primary amine(s);
h) formation of an amide formation of the by reaction of the subsequent terminal amine and a carboxylic end of a linker of the structure: 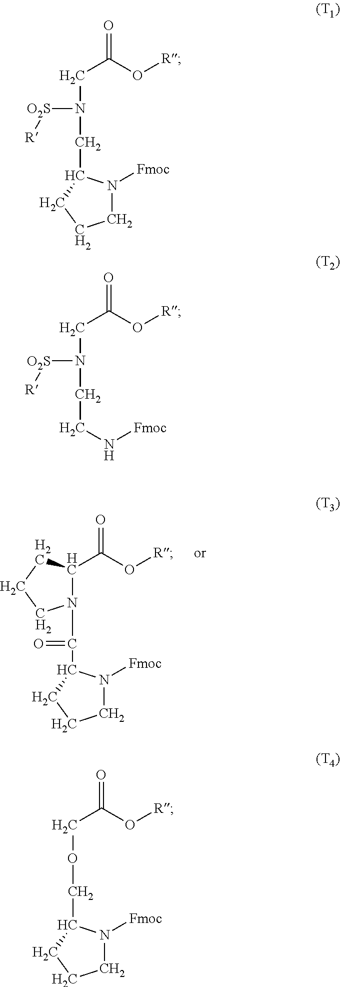
wherein where R′ is an organic group and R″ is H or a carboxylic acid protecting group and, subsequently deprotecting an amine of the linker by the removal of an Fmoc group to form a subsequent terminal amine, or repeating steps d) and e) twice;
i) repeating step d);
j) repeating steps c) through g);
k) reacting the subsequent terminal amine and a carboxylic end of the substrate bound linker to form a cyclic; and
l) removing the 2,4-dimethoxybenzyl (DMB) groups and any other protecting groups to yield a substrate bound peptoid body.

16. The method according to claim 15, wherein the primary amine is RNH₂ wherein R is C₁-C₁₂ alkyl, C₁-C₁₂ hydroxyalkyl, C₁-C₁₂ aminoalkyl, C₁-C₁₂ carboxylic acid alkyl, C₂-C₁₂ alkyloxyalkyl, C₂-C₁₂ alkenyl, C₂-C₁₂ hydroxyalkenyl, C₂-C₁₂ aminoalkenyl, C₁-C₁₂ carboxylic acid alkenyl, C₃-C₁₄ alkyloxyalkenyl, C₆-C₁₄ aryl, C₆-C₁₄ hydroxyaryl, C₆-C₁₄ aminoaryl, C₆-C₁₄ carboxylic acid aryl, C₇-C₁₅ alkyloxyaryl, C₄-C₁₄ heteroaryl, C₄-C₁₄ hydroxyheteroaryl, C₄-C₁₄ aminoheteroaryl, C₄-C₁₄ carboxylic acid heteroaryl, C₅-C₁₅ alkoxyheteroaryl, C₇-C₁₅ alkylaryl, C₇-C₁₅ hydroxyalkylaryl, C₇-C₁₅ aminoalkylaryl, C₇-C₁₅ carboxylic acid alkylaryl, C₈-C₁₅ alkoxyalkylaryl, or any chemically transformed structure therefrom.

17. The method according to claim 15, wherein the primary amine is 4-aminopiperidine; ethanolamine; allylamine; 1;4-diaminobutane; piperponylamine; 4; (2-aminoethyl)benzene; isobutylamine; tryptamine; 4-morpholinoaniline; 5-amino-2-methoxypyridine; (R)-methylbenzylamine; 1-(2-aminopropyl)-2-pyrrolidinone; furfurylamine; benzylamine; 4-chlorobenzylamine; 4-methoxybenzylamine; methoxyethylamine. 2-aminoadipic acid; N-ethylasparagine; 3-aminoadipic acid; hydroxylysine; beta-alanine; allo-hydroxylysine propionic acid; 2-aminobutyric acid; 3-hydroxyproline; 4-Aminobutyric acid; 4-hydroxyproline piperidinic acid; 6-Aminocaproic acid; Isodesmosine; 2-Aminoheptanoic acid; allo-isoleucine; 2-aminoisobutyric acid; N-methylglycine; 3-aminoisobutyric acid; N-methylisoleucine; 2-Aminopimelic acid; 6-N-methyllysine; 2,4-diaminobutyric acid; N-methylvaline; desmosine; norvaline; 2,2′-diaminopimelic acid; norleucine; 2,3-diaminopropionic acid; ornithine; N-ethylglycine; or any protected equivalents thereof.

18. A method of identifying an inhibiting peptoid-body, comprising contacting an array of peptoid-bodies according to claim 12 with a sample comprising a target; and identifying where binding to one or more peptoid-bodies to the target is indicated.

19. The method of claim 18, wherein said contacting is carried out on a substrate, wherein the target comprises cells and/or proteins, and wherein said identifying comprises identifying a well on the substrate where binding to target in the sample is indicated.

20. A pharmaceutical composition, comprising a peptoid-body according to claim 1.

21. The pharmaceutical composition according to claim 20, further comprising one or more other anti-cancer agents.

22. A method for treating a disorder in a subject, comprising administering an effective amount of a pharmaceutical composition according to claim 20 to the subject.

23. The method according to claim 22, wherein the disorder is an oncological disorder, infection, or immunoregulatory disorder.

24. The method according to claim 22, wherein the disorder is an autoimmune or chronic inflammatory disease.

25. A method for inducing apoptosis or inhibiting the growth of a cell, comprising contacting the cell with an effective amount of a peptoid body in vitro or in vivo according to claim 1.

26. The method according to claim 25, wherein the cell is a cancer cell or a pathogen-infected cell.
